(19) United States
(12) Patent Application Publication
(10) Pub. No.: US 2010/0136143 A1
(43) Pub. Date: Jun. 3, 2010

(54) NEW COMPOSITIONS AND METHODS FOR CELL KILLING

(76) Inventor: Shmuel Bukshpan, Ramat Ha-Shshon (IL)

Correspondence Address: Fleit Gibbons Gutman Bongini & Bianco PL, 21355 EAST DIXIE HIGHWAY, SUITE 115, MIAMI, FL, 33180, US

(21) Appl. No.: 12/594,384

(22) PCT Filed: Apr. 3, 2008
(86) PCT No.: PCT/IL08/00465

§ 371 Date: Oct. 2, 2009

(30) Foreign Application Priority Data
Apr. 3, 2007 (US) 60907463

(51) Int. Cl. A61K 33/00; A61K 31/16; C12N 5/00

(52) U.S. Cl. 424/724; 514/616; 435/375

(57) ABSTRACT

The present invention discloses an insoluble proton sink or source (PSS), useful for killing living target cells (LTCs), or otherwise disrupting vital intracellular processes and/or intercellular interactions of the LTC upon contact. The PSS comprises (i) proton source or sink providing a buffering capacity; and (ii) means providing proton conductivity and/or electrical potential. The PSS is effectively disrupting the pH homeostasis and/or electrical balance within the confined volume of the LTC and/or disrupting vital intercellular interactions of the LTCs while efficiently preserving the pH of the LTCs' environment. The invention also provides articles of manufacture comprises the PSS and presents an effective method for killing the LTCs.

### FIELD OF THE INVENTION

The present invention pertains to compositions and methods for killing cells. More specifically, to compositions and methods for killing living target cells, or otherwise disrupting vital intracellular processes and/or intercellular interactions of the cells, while efficiently preserving the pH of the cells environment.

### FIELD AND BACKGROUND OF THE INVENTION

Various forms of cellular material are known to be harmful and potentially lethal to man. For example, cancerous cells are the second leading cause of death in the United States, after heart disease (Boring et al., (1993), CA Cancer Journal for Clinicians 43:7). Cellular microorganisms are also responsible for a wide range of diseases. Targeted and selective cell killing (e.g., cancer cells and pathogenic bacteria) is extensively investigated in the biotechnology industry.

Microorganisms can invade the host tissues and proliferate, causing severe disease symptoms. Pathogenic bacteria have been identified as a root cause of a variety of debilitating or fatal diseases including, for example, tuberculosis, cholera, whooping cough, plague, and the like. To treat such severe infections, drugs such as antibiotics are administered that kill the infectious agent. However, pathogenic bacteria commonly develop resistance to antibiotics and improved agents are needed to prevent the spread of infections due to such microorganisms.

Infection is a frequent complication of many invasive surgical, therapeutic and diagnostic procedures. For procedures involving implantable medical devices, avoiding infection can be particularly problematic because bacteria can develop into biofilms, which protect the microbes from clearing by the subject's immune system. As these infections are difficult to treat with antibiotics, removal of the device is often necessitated, which is traumatic to the patient and increases the medical cost.

Since the difficulties associated with eliminating biofilm-based infections are well recognized, a number of technologies have developed to treat surfaces or fluids bathing surfaces to prevent or impair biofilm formation. Biofilms adversely affect medical systems and other systems essential to public health such as water supplies and food production facilities. A number of technologies have been proposed that treat surfaces with organic or inorganic materials to interfere with biofilm development. For example, various methods have been employed to coat the surfaces of medical devices with antibiotics (See e.g., U.S. Pat. Nos. 4,107,121, 4,442,133, 4,895,566, 4,917,686, 5,013,306, 4,952,419, 5,853,745 and 5,902,283) and other bacteriostatic compounds (See e e.g., U.S. Pat. Nos. 4,605,564, 4,886,505, 5,019,096, 5,295,979, 5,328,954, 5,681,575, 5,753,251, 5,770,255, and 5,877,243).

Despite these technologies, contamination of medical devices and invasive infection therefrom continues to be a problem.

Infectious organisms are ubiquitous in the medical environment, despite vigorous efforts to maintain antisepsis. The presence of these organisms can result in infection of hospitalized patients and medical personnel. These infections, termed nosocomial, often involve organisms more virulent and more unusual than those encountered outside the hospital. In addition, hospital-acquired infections are more likely to involve organisms that have developed resistance to a number of antibiotics. Although cleansing and anti-bacterial regimens are routinely employed, infectious organisms readily colonize a variety of surfaces in the medical environment, especially those surfaces exposed to moisture or immersed in fluid. Even barrier materials, such as gloves, aprons and shields, can spread infection to the wearer or to others in the medical environment. Despite sterilization and cleansing, a variety of metallic and non-metallic materials in the medical environment can retain dangerous organisms trapped in a biofilm, thence to be passed on to other hosts.

Any agent used to impair biofilm formation in the medical environment must be safe to the user. Certain biocidal agents, in quantities sufficient to interfere with biofilms, also can damage host tissues. Antibiotics introduced into local tissue areas can induce the formation of resistant organisms which can then form biofilm communities whose planktonic microorganisms would likewise be resistant to the particular antibiotics. Any anti-biofilm or antifouling agent must furthermore not interfere with the salubrious characteristics of a medical device. Certain materials are selected to have a particular type of operator manipulability, softness, water-tightness, tensile strength or compressive durability, characteristics that cannot be altered by an agent added for anti-microbial effects. As a further problem, it is possible that materials added to the surfaces of implantable devices to inhibit contamination and biofilm formation may be thrombogenic.

Biofilm formation has important public health implications. Drinking water systems are known to harbor biofilms, even though these environments often contain disinfectants. Any system providing an interface between a surface and a fluid has the potential for biofilm development. Water cooling towers for air conditioners are well-known to pose public health risks from biofilm formation, as episodic outbreaks of infections like Legionnaires' disease attest. Biofilms have been identified in flow conduits like hemodialysis tubing, and in water distribution conduits. Biofilms have also been identified to cause biofouling in selected municipal water storage tanks, private wells and drip irrigation systems, unaffected by treatments with up to 200 ppm chlorine.

Biofilms are a constant problem in food processing environments. Food processing involves fluids, solid material and their combination. As an example, milk processing facilities provide fluid conduits and areas of fluid residence on surfaces. Cleansing milking and milk processing equipment presently utilizes interactions of mechanical, thermal and chemical processes in air-injected clean-in-place methods. Additionally, the milk product itself is treated with pasteurization. In cheese producing, biofilms can lead to the production of calcium lactate crystals in Cheddar cheese. Meat processing and packing facilities are in like manner susceptible to biofilm formation. Non-metallic and metallic surfaces can be affected. Biofilms in meat processing facilities have been detected on rubber “fingers,” plastic curtains, conveyor belt material, evisceration equipment and stainless steel surfaces. Controlling biofilms and microorganism contamination in food processing is hampered by the additional need that the agent used not affects the taste, texture or aesthetics of the product.

There exists, therefore, a need to be able to render general surfaces bactericidal. There is a keen interest in materials capable of killing harmful microorganisms. Such materials could be used to coat surfaces of common objects touched by people in everyday lives, e.g., door knobs, children toys, computer keyboards, telephones, fabrics, medical devices etc., to render them antiseptic and thus unable to transmit bacterial infections. Since ordinary materials are not antimicrobial or cell-killing, their modification is required. For example, surfaces chemically modified with poly(ethylene glycol) and certain other synthetic polymers can repel (although not kill) microorganisms (Bridgett, M. J., et al., (1992) Biomaterials 13,411-416; Arciola, C. R., et al Alvergna, P., Cenni, E. & Pizzoferrato, A. (1993) Biomaterials 14,1161-1164; Park, K. D., Kim, Y. S., Han, D. K., Kim, Y. H., Lee, E. H. B., Suh, H. & Choi, K. S. (1998) Biomaterials 19, 51-859.).

Alternatively, materials can be impregnated with antimicrobial agents, such as antibiotics, quarternary ammonium compounds, silver ions, or iodine, that are gradually released into the surrounding solution over time and kill deleterious cells and microorganisms there (Medlin, J. (1997) Environ. Health Preps. 105,290-292; Nohr, R. S. & Macdonald, G. J. (1994) J. Biomater. Sci., Polymer Edn. 5,607-619 Shearer, A. E. H., et al (2000) Biotechnol. Bioeng 67,141-146.). Although these strategies have been verified in aqueous solutions containing bacteria, they would not be expected to be effective against airborne bacteria in the absence of a liquid medium; this is especially true for release-based materials, which are also liable to become impotent when the leaching antibacterial agent is exhausted.

General surface coating/derivatization procedures have been developed that should be applicable to most materials regardless of their nature.

There exist polymers with inherent antimicrobial or antistatic properties. Such polymers can be applied or used in conjunction with a wide variety of substrates (e.g., glass, textiles, metal, cellulosic materials, plastics, etc.) to provide the substrate with antimicrobial and/or antistatic properties. In addition, the polymers can also be combined with other polymers to provide such other polymers with antimicrobial and/or antistatic properties.

However, there is also a need for such agents to, be both sustainable and to be compatible, and to be used on and with a wide variety of polymer materials and substrates. Various additives and polymer systems have been suggested as providing antimicrobial properties. See, for example, U.S. Pat. No. 3,872,128 to Byck, U.S. Pat. No. 5,024,840 to Blakely et al, U.S. Pat. No. 5,290,894 to Malrose et al, U.S. Pat. Nos. 5,967,714, 6,203,856 and U.S. Pat. No. 6,248,811 to Ottersbach et al, U.S. Pat. No. 6,194,530 to Klasse et al. and U.S. U.S. patent to Siddiqui et al.

There, however, remains a need for potentially less toxic polymer compositions that provide sustainable cell killing properties to a wide variety of substrates and materials.

It is quite well known that charged molecules in solution are able to kill bacteria (Endo et al., 1987; Fidai et al., 1997; Friedrich et al., 2000; Isquith et al., 1972). However, it has been realized more recently that charges attached to surfaces can kill bacteria upon contact. All bear cationic, positively charged groups, such as quaternary ammonium (Thome et al., 2003) or phosphonium (Kanazawa et al., 1993; Popa et al., 2003). Various architectures have been tested: self-assembled monolayers (Atkins, 1990; Gottenbos et al., 2002; Rondelez & Bezou, 1999), polyelectrolyte layers (Lee et al., 2004; Lin et al., 2002, 2003; Popa et al., 2003; Sauvet et al., 2000; Thome et al., 2003; Tiller et al., 2001) and hyperbranched dendrimers (Cen et al., 2003; Chen & Cooper, 2000, 2002). An important advantage of this approach is that the biocidal molecules are attached covalently to the substrates, which allows their reusability after cleaning processes and prevents uncontrolled material release to the environment. However, the key parameters of the effects involved in the biocidal process have not yet been identified.

Recently, Kügler et al, (2005) reported on the existence of a charge-density threshold above which bacterial death occurs quickly upon adsorption on substrates bearing cationic quaternary ammonium groups. The authors have grafted quaternized poly(vinylpyridine) chains on glass surfaces by two different methods and varied the outer-layer charge density (OLCD) within the organic layer between 10¹² and 10¹⁶ positive charges per cm². Their measurements showed that this parameter has a large influence on the killing efficiency. Bacterial death occurs in less than 10 min in the quiescent state above a threshold value. The OLCD values were 10 to 100-fold smaller for bacteria in the growth state. It also depends on the bacteria type, and they have observed a difference by a factor of 10 between Escherichia coli and Staphylococcus epidermidis under high-division conditions. Based on their results, these authors proposed a cell-killing mechanism based on ion exchange between the bacterial membrane and the functionalized surface.

Nevertheless, all of the above described publications and U.S. patent applications teach the effect of surface treatment, surface properties and the crucial necessity for close surface contact in order to kill cells. None of the above teaches the “BULK EFFECT” of Solid acidic or basic proton/hydroxyl conductors and buffers on live cells. Nor do they teach the use of barrier layers coating of cytotoxic polymers as a way of killing cells. Furthermore, none of the above mentioned US patent applications teach configuration of the polymers to selectively kill certain cell types.

There thus remains a need for and it would be highly advantageous to have agents capable of sustained and long-acting cytotoxic action both against eukaryotic and prokaryotic cells. On way of achieving these desirable goals is by coating solid ion exchangers (SIEx) with coating materials which will, in general, be with diffusion barrier properties and with no inherent pharmacological or toxic properties. These coating materials will limit the transport of competing counter-ions to/from the SIEx surface yet allowing proton/hydroxyl ions transfer. Thereby, eliminating or substantially reducing the ion-exchange saturation by counter-ions, resulting in sustained and long acting activity thereof.

U.S. Pat. No. 6,001,392, to Wen et al. describes the use of a mixture of coated and non-coated sulfonic acid cation exchange resins (Amberlite™ IRP-69; (obtained from Rohm and Haas) cross-linked with divinyl benzene onto which dextromethorphan (a commonly used antitussive drug) has been loaded to provide a sustained-release of the drug in a liquid formulation. About 30% of the drug/resin complexes are coated with a mixture of ethyl cellulose or ethyl cellulose latexes with plasticizers and water dispersible polymers such as SURELEASE. Other examples of resins and coating materials described for the above mentioned purpose are: Dow XYS-40010.00 (Dow Chemical Company). Amberlite™ IRP-69 and Dow XYS-40010.00 are both sulfonated polymers composed of polystyrene cross-linked with 8% of divinylbenzene, with an ion exchange capacity of about 4.5 to 5.5 meq./g of dry resin (H⁺-form). Their essential difference is in physical form. Amberlite™ IRP-69 consists of irregularly-shaped particles with a size range of 47 to 149 um, produced by milling the parent, large-sized spheres of Amberlite™ IRP-120. The Dow XYS-40010.00 product consists of spherical particles with a size range of 45 to 150 um. Another useful exchange resin, Dow XYS-40013.00, is a polymer composed of polystyrene cross-linked with 8% of divinylbenzene and functionalized with a quaternary ammonium group; its exchange capacity is normally within the range of approximately 3 to 4 meq./g of dry resin.

The coating materials can in general be any of a large number of conventional natural or synthetic film-forming materials used singly, in admixture with each other, and in admixture with plasticizers, pigments, etc. with diffusion barrier properties and with no inherent pharmacological or toxic properties. In general, the major components of the coating should be insoluble in water and permeable to water. However, it might be desirable to incorporate a water-soluble substance, such as methyl cellulose, to alter the permeability of the coating, or to incorporate an acid-insoluble, base-soluble substance to act as an enteric coating. The coating materials may be applied as a suspension in an aqueous fluid or as a solution in organic solvents. Suitable examples of such coating materials are described by R. C. Rowe in Materials used in Pharmaceutical Formulation. (A. T. Florence, editor), Blackwell Scientific Publications, Oxford, 1-36(1984), incorporated by reference herein. The water-permeable diffusion barrier is selected from the group consisting of ethyl cellulose, methyl cellulose and mixtures thereof an example of which is SURELEASE, manufactured by Colorcon, which is water based ethyl cellulose latex, plasticized with dibutyl sebacate or with vegetable oils. Other non-limiting coating materials are AQUACOAT, manufactured by FMC Corporation of Philadelphia, which is ethylcellulose pseudolatex; solvent based ethylcellulose; shellac; zein; rosin esters; cellulose acetate; EUDRAGITS, manufactured by Rohm and Haas of Philadelphia, which are acrylic resins; silicone elastomers; poly(vinyl chloride) methyl cellulose; and hydroxypropylmethyl cellulose.

Conventional coating solvents and coating procedures (such as fluid bed coating and spray coating) can be employed to coat the particles. Techniques of fluid bed coating are taught, for example, in U.S. Pat. Nos. 3,089,824; 3,117,027; and 3,253,944. Non-limiting examples of coating solvents include ethanol, a methylene chloride/acetone mixture, coating emulsions, methyl acetone, tetrahydrofuran, carbonetetrachloride, methyl ethyl ketone, ethylene dichloride, trichloroethylene, hexane, methyl alcohol, isopropyl alcohol, methyl isobutyl ketone, toluene, 2-nitropropane, xylene, isobutyl alcohol, n-butyl acetate. The above described art can be applied in the present invention to design coated SIEs for selective and targeted cell killing purposes.

It is well established that extreme pH values in solution (high above 7.0 and below 5.5) are harmful to cells (microbial as well as mammalian) therefore, rendering a cytotoxic effect. GB Pat. No. 2374287 to Bennett et al., describes a composition for sanitizing and/or disinfecting a non-porous hard surface comprises of an alcohol selected from the group consisting of methanol, ethanol, n-propanol, isopropanol, n-butanol, benzyl alcohol, and mixtures thereof which is present in an amount of from about 40 to about 70 weight percent and an effective amount of a pH modifying agent such that the pH range of the composition is from about 7.0 to about 13.0, wherein the amount of alcohol is inversely proportional to the pH of the composition. It has been found that by increasing the pH of the composition lower amounts of alcohol can be used. Thus effective disinfecting compositions have been provided with reduced VOC (volatile organic compound) content. On the other hand, U.S. Pat. No. 5,614,241 to Woodrow, describes a nutritionally balanced water soluble powdered food composition which, when mixed with water, has a low pH (between 5.5. to 2.0), extended shelf life, high antimicrobial activity, and which includes protein alpha-amino acids in solution or in suspension. The food composition utilizes a low pH binary protein stabilizer system and a high total acidity-low pH bacteria stabilizer system.

However, the above mentioned patents and other previous arts, teaches the antimicrobial effect of pH in liquid solutions. None of the above neither demonstrates nor teaches the cytotoxic effect of solid buffers and ion exchangers through proton exchange between the cell-membrane and the ion exchange, without adversely affecting the pH of the solution.

The following publications are incorporated hereinafter as a reference: Arciola, C. R., Alvergna, P., Cenni, E. & Pizzoferrato, A. (1993) Biomaterials 14, 1161-1164. Atkins, P. W. (1990). Physical Chemistry. New York: W. H. Freeman & Company. Boring et al., CA Cancer Journal for Clinicians. 43:7 1993. Bridgett, M. J., et al., (1992). Biomaterials 13, 411-416. Cen, L., Neoh, K. G. & Kang, E. T. (2003). Langmuir 19, 10295-10303. Chen, C. Z. & Cooper, S. L. (2000). Adv Materials 12, 843-846. Chen, C. Z. & Cooper, S. L. (2002). Biomaterials 23, 3359-3368. Endo, Y., Tani, T. & Kodama, M. (1987). Appl Environ Microbiol 53, 2050-2055. Fidai, S., Farer, S. W. & Hancock, R. E. (1997). Methods Mol Biol 78, 187-204. Friedrich, C. L., Moyles, D., Beverige, T. J. & Hancock, R. E. W. (2000). Antimicrob Agents Chemother 44, 2086-2092. Gottenbos, B., van der Mei, H. C., Klatter, F., Nieuwenhuis, P. & Busscher, H. J. (2002). Biomaterials 23, 1417-1423. Isquith, A. J., Abbott, E. A. & Walters, P. A. (1972). Appl Microbiol 24, 859-863. Kanazawa, A., Ikeda, T. & Endo, T. (1993). J Polym Sci Part A Polym Chem 31, 1467-1472. Kügler R., Bouloussa O. and Rondelez F., (2005) Microbiology, 151, 1341-1348. Lee, S. B., Koepsel, R. R., Morley, S. W., Matyajaszewski, K., Sun, Y. & Russell, A. J. (2004). Biomacromolecules 5, 877-882. Lin, J., Qiu, S., Lewis, K. & Klibanov, A. M. (2002). Biotechnol Prog 18, 1082-1096. Lin, J., Qiu, S., Lewis, K. & Klibanov, A. M. (2003). Biotechnol Bioeng 83, 168-172. Medlin J. 1997. Germ warfare. Environ Health Persp 105:290-292. Nohr R S and Macdonald G J. 1994. J Biomater Sci, Polymer Edn 5:607-619. Park, K. D., Kim, Y. S., Han, D. K., Kim, Y. H., Lee, E. H. B., Suh, H. & Choi, K. S. (1998) Biomaterials 19, 51-859. Popa, A., Davidescu, C. M., Trif, R., Ilia, Gh., Iliescu, S. & Dehelean, Gh. (2003). React Funct Polym 55, 151-158. Rondelez, F. & Bezou, P. (1999). Actual Chim 10, 4-8. Rowe R. C. (1984) in Materials used in Pharmaceutical Formulation. (A. T. Florence, editor), Blackwell Scientific Publications, Oxford, 1-36. Shearer, A. E. H., et al., (2000), Biotechnol. Bioeng 67,141-146. Sauvet, G., Dupond, S., Kazmierski, K. & Chojnowski, J. (2000). J Appl Polym Sci 75, 1005-1012. Thome, J., Holländer, A., Jaeger, W., Trick, I. & Oehr, C. (2003). Surface Coating Technol 174-175, 584-587. Tiller, J. C., Liao, C., Lewis, K. & Klibanov, A. M. (2001). Proc Natl Acad Sci USA 98, 5981-5985.

### SUMMARY OF THE INVENTION

It is hence one object of the invention to disclose an insoluble proton sink or source (PSS), useful for killing living target cells (LTCs), or otherwise disrupting vital intracellular processes and/or intercellular interactions of the LTC upon contact. The PSS comprising (i) proton source or sink providing a buffering capacity; and (ii) means providing proton conductivity and/or electrical potential; wherein said PSS is effectively disrupting the pH homeostasis and/or electrical balance within the confined volume of the LTC and/or disrupting vital intercellular interactions of the LTCs while efficiently preserving the pH of the LTCs' environment.

It is in the scope of the invention wherein the PSS is an insoluble hydrophobic, either anionic, cationic or zwitterionic charged polymer, useful for killing living target cells (LTCs), or otherwise disrupting vital intracellular processes and/or intercellular interactions of the LTC upon contact. It is additionally or alternatively in the scope of the invention, wherein the PSS is an insoluble hydrophilic, anionic, cationic or zwitterionic charged polymer, combined with water-immiscible polymers useful for killing living target cells (LTCs), or otherwise disrupting vital intracellular processes and/or intercellular interactions of the LTC upon contact. It is further in the scope of the invention, wherein the PSS is an insoluble hydrophilic, either anionic, cationic or zwitterionic charged polymer, combined with water-immiscible either anionic, cationic of zwitterionic charged polymer useful for killing living target cells (LTCs), or otherwise disrupting vital intracellular processes and/or intercellular interactions of the LTC upon contact.

It is also in the scope of the invention wherein the PSS is adapted in a non-limiting manner, to contact the living target cell either in a bulk or in a surface; e.g., at the outermost boundaries of an organism or inanimate object that are capable of being contacted by the PSS of the present invention; at the inner membranes and surfaces of microorganisms, animals and plants, capable of being contacted by the PSS by any of a number of transdermal delivery routes etc; at the bulk, either a bulk provisioned with stirring or nor etc.

It is further in the scope of the invention wherein either (i) a PSS or (ii) an article of manufacture comprising the PSS also comprises an effective measure of at least one additive.

It is another object of the invention to disclose the PSS as defined in any of the above, wherein the proton conductivity is provided by water permeability and/or by wetting, especially wherein the wetting is provided by hydrophilic additives.

It is another object of the invention to disclose the PSS as defined in any of the above, wherein the proton conductivity or wetting is provided by inherently proton conductive materials (IPCMs) and/or inherently hydrophilic polymers (IHPs), especially by IPCMs and/or IHPs selected from a group consisting of sulfonated tetrafluortheylene copolymers; sulfonated materials selected from a group consisting of silica, polythion-ether sulfone (SPTES), styrene-ethylene-butylene-styrene (S-SEBS), polyether-ether-ketone (PEEK), poly(arylene-ether-sulfone) (PSU), Polyvinylidene Fluoride (PVDF)-grafted styrene, polybenzimidazole (PBI) and polyphosphazene; proton-exchange membrane made by casting a polystyrene sulfonate (PSSnate) solution with suspended micron-sized particles of cross-linked PSSnate ion exchange resin; commercially available Nafion™ and derivatives thereof.

It is another object of the invention to disclose the PSS as defined in any of the above, wherein the PSS is constructed as a conjugate, comprising two or more, either two-dimensional (2D) or three-dimensional (3D) PSSs, each of which of the PSSs consisting of materials containing highly dissociating cationic and/or anionic groups (HDCAs) spatially organized in a manner which efficiently minimizes the change of the pH of the LTC's environment. Each of the HDCAs is optionally spatially organized in specific either 2D, topologically folded 2D surfaces, or 3D manner efficiently which minimizes the change of the pH of the LTC's environment; further optionally, at least a portion of the spatially organized HDCAs are either 2D or 3D positioned in a manner selected from a group consisting of (i) interlacing; (ii) overlapping; (iii) conjugating; (iv) either homogeneously or heterogeneously mixing; and (iv) tiling the same.

It is acknowledged in this respect to underline that the term HDCAs refers, according to one specific embodiment of the invention, and in a non-limiting manner, to ion-exchangers, e.g., water immiscible ionic hydrophobic materials.

It is another object of the invention to disclose the PSS as defined in any of the above, wherein the PSS is effectively disrupting the pH homeostasis within a confined volume while efficiently preserving the entirety of the LTC's environment; and further wherein the environment's entirety is characterized by parameters selected from a group consisting of the environment functionality, chemistry; soluble's concentration, possibly other then proton or hydroxyl concentration; biological related parameters; ecological related parameters; physical parameters, especially particles size distribution, rehology and consistency; safety parameters, especially toxicity, otherwise LD₅₀ or ICT₅₀ affecting parameters; olphactory or organoleptic parameters (e.g., color, taste, smell, texture, conceptual appearance etc); or any combination of the same.

It is another object of the invention to disclose the PSS as defined in any of the above, wherein the PSS is provided useful for disrupting vital intracellular processes and/or intercellular interactions of the LTC, while both (i) effectively preserving the pH of the LTC's environment and (ii) minimally affecting the entirety of the LTC's environment such that a leaching from the. PSS of either ionized or neutral atoms, molecules or particles (AMP) to the LTC's environment is minimized.

It is well in the scope of the invention wherein the aforesaid leaching minimized such that the concentration of leached ionized or neutral atoms is less than 1 ppm. Alternatively, the aforesaid leaching is minimized such that the concentration of leached ionized or neutral atoms is less than less than 50 ppb. Alternatively, the aforesaid leaching is minimized such that the concentration of leached ionized or neutral atoms is less than less than 50 ppb and more than 10 ppb. Alternatively, the aforesaid leaching is minimized such that the concentration of leached ionized or neutral atoms is less than less than 10 but more than 0.5 ppb. Alternatively, the aforesaid leaching is minimized such that the concentration of leached ionized or neutral atoms is less than less than 0.5 ppb.

It is another object of the invention to disclose the PSS as defined in any of the above, wherein the PSS is provided useful for disrupting vital intracellular processes and/or intercellular interactions of the LTC, while less disrupting pH homeostasis and/or electrical balance within at least one second confined volume (e.g., non-target cells, NTC).

It is another object of the invention to disclose the differentiating PSS as defined in any of the above, wherein differentiation between the LTC and NTC is obtained by one or more of the following means: (i) providing differential ion capacity; (ii) providing differential pH values; and, (iii) optimizing PSS to target cell size ratio; (iv) providing a differential spatial, either 2D, topologically folded 2D surfaces, or 3D configuration of the PSS; (v) providing a critical number of PSS' particles (or applicable surface) with a defined capacity per a given volume; and (vi) providing size exclusion means.

It is another object of the invention to disclose an article of manufacture, comprising at least one insoluble non-leaching PSS as defined in any of the above. The PSS, located on the internal and/or external surface of the article, is provided useful, upon contact, for disrupting pH homeostasis and/or electrical balance within at least a portion of an LTC while effectively preserving pH & functionality of the surface.

It is another object of the invention to disclose an article of manufacture, comprising at least one insoluble non-leaching PSS as defined in any of the above. Especially adapted for target cell's killing. The PSS is having at least one external proton-permeable surface with a given functionality (e.g., electrical current conductivity, affinity, selectivity etc), the surface is at least partially composed of, or topically and/or underneath layered with a PSS, such that disruption of vital intracellular processes and/or intercellular interactions of the LTC is provided, while the LTC's environment's pH & the functionality is effectively preserved.

It is another object of the invention to disclose an article of manufacture, comprising at least one insoluble non-leaching PSS as defined in any of the above, comprising a surface with a given functionality, and one or more external proton-permeable layers, each of which of the layers is disposed on at least a portion of the surface; wherein the layer is at least partially composed of or layered with a PSS such that vital intracellular processes and/or intercellular interactions of the LTC are disrupted, while the LTC's environment's pH & the functionality is effectively preserved.

It is another object of the invention to disclose an article of manufacture, comprising at least one insoluble non-leaching PSS as defined in any of the above. The PSS-based system comprising (i) at least one PSS; and (ii) one or more preventive barriers, providing the PSS with a sustained long activity; preferably wherein at least one barrier is a polymeric preventive barrier adapted to avoid heavy ion diffusion; further preferably wherein the polymer is an ionomeric barrier, and particularly a commercially available Nafion™).

It is acknowledged in this respect that the presence or incorporation of barriers that can selectively allow transport of protons and hydroxyls but not of other competing ions to and/or from the SIEx surface eliminates or substantially reduces the ion-exchange saturation by counter-ions, resulting in sustained and long acting cell killing activity of the materials and compositions of the current invention.

It is in the scope of the invention, wherein the proton and/or hydroxyl-exchange between the cell and strong acids and/or strong basic materials and compositions may lead to disruption of the cell pH-homeostasis and consequently to cell death. The proton conductivity property, the volume buffer capacity and the bulk activity are pivotal and crucial to the present invention.

It is further in the scope of the invention, wherein the pH derived cytotoxicity can be modulated by impregnation and coating of acidic and basic ion exchange materials with polymeric and/or ionomeric barrier materials.

It is another object of the invention to disclose an article of manufacture, comprising at least one insoluble non-leaching PSS as defined in any of the above, adapted to avoid development of LTC's resistance and selection over resistant mutations.

It is another object of the invention to disclose an article of manufacture as defined in any of the above, designed and constructed as a member of a group consisting of barriers; membranes; filers; pads; meshes; nets; inserts; particulate matter; powders, nano-powders and the like; vehicles, carriers or vesicles consisting a PSS (e.g., liposomes with PSSs); doped, coated, immersed, contained, soaked, immobilized, entrapped, affixed, set in a column, solubilized, or otherwise bonded PSS-containing matter.

It is another object of the invention to disclose an article of manufacture, characterized by at least one of the following (i) regeneratable proton source or sink; (ii) regeneratable buffering capacity; and (iii) regeneratable proton conductivity.

It is another object of the invention to disclose a method for killing living target cells (LTCs), or otherwise disrupting vital intracellular processes and/or intercellular interactions of the LTC upon contact. The method comprising steps of providing at least one PSS having (i) proton source or sink providing a buffering capacity; and (ii) means providing proton conductivity and/or electrical potential; contacting the LTCs with the PSS; and, by means of the PSS, effectively disrupting the pH homeostasis and/or electrical balance within the LTC while efficiently preserving the pH of the LTC's environment.

It is another object of the invention to disclose a method as defined above, wherein the aforthee first step further comprising a step of providing the PSS with water permeability and/or wetting characteristics, in particular wherein the proton conductivity and wetting is at least partially obtained by providing the PSS with hydrophilic additives.

It is another object of the invention to disclose a method as defined above, wherein the method further comprising a step of providing the PSS with inherently proton conductive materials (IPCMs) and/or inherently hydrophilic polymers (IHPs), especially by selecting the IPCMs and/or IHPs from a group consisting of sulfonated tetrafluoroetheylene copolymers; commercially available Nafion™ and derivatives thereof.

It is another object of the invention to disclose a method as defined above, wherein the method further comprising steps of providing two or more, either two-dimensional (2D), topologically folded 2D surfaces, or three-dimensional (3D) PSSs, each of which of the PSSs consisting of materials containing highly dissociating cationic and/or anionic groups (HDCAs); and, spatially organizing the HDCAs in a manner which minimizes the change of the pH of the LTC's environment.

It is another object of the invention to disclose a method as defined above, wherein the method further comprising a step of spatially organizing each of the HDCAs in a specific, either 2D or 3D manner, such that the change of the pH of the LTC's environment is minimized.

It is another object of the invention to disclose a method as defined above, wherein the step of organizing is provided by a manner selected for a group consisting of (i) interlacing the HDCAs; (ii) overlapping the HDCAs; (iii) conjugating the HDCAs; (iv) either homogeneously or heterogeneously mixing the HDCAs; and (v) tiling of the same.

It is another object of the invention to disclose a method as defined above, wherein the method further comprising a step of disrupting pH homeostasis and/or electrical potential within at least a portion of an LTC by a PSS, while both (i) effectively preserving the pH of the LTC's environment; and (ii) minimally affecting the entirety of the LTC's environment; the method is especially provided by minimizing the leaching of either ionized or electrically neutral atoms, molecules or particles from the PSS to the LTC's environment.

It is another object of the invention to disclose a method as defined above, wherein the method further comprising steps of preferentially disrupting pH homeostasis and/or electrical balance within at least one first confined volume (e.g., target living cells, LTC), while less disrupting pH homeostasis within at least one second confined volume (e.g., non-target cells, NTC).

It is another object of the invention to disclose the differentiating method as defined above, wherein the differentiation between the LTC and NTC is obtained by one or more of the following steps: (i) providing differential ion capacity; (ii) providing differential pH value; (iii) optimizing the PSS to LTC size ratio; and, (iv) designing a differential spatial configuration of the PSS boundaries on top of the PSS bulk; and (v) providing a critical number of PSS' particles (or applicable surface) with a defined capacity per a given volume; and (vi) providing size exclusion means, e.g., mesh, grids etc.

It is another object of the invention to disclose a method for the production of an article of manufacture, comprising steps of providing an PSS as defined above; locating the PSS on top or underneath the surface of the article; and upon contacting the PSS with an LTC, disrupting the pH homeostasis and/or electrical balance within at least a portion of the LTC while effectively preserving pH & functionality of the surface.

It is another object of the invention to disclose a method as defined above, wherein the method further comprising steps of providing at least one external proton-permeable surface with a given functionality; providing at least a portion of the surface with at least one PSS, and/or layering at least one PSS on top of, or underneath the surface; hence killing LTCs or otherwise disrupting vital intracellular processes and/or intercellular interactions of the LTC, while effectively preserving the LTC's environment's pH & functionality.

It is another object of the invention to disclose a method as defined above, wherein the method further comprising steps of providing at least one external proton-permeable providing a surface with a given functionality; disposing one or more external proton-permeable layers topically and/or underneath at least a portion of the surface; the one or more layers are at least partially composed of or layered with at least one PSS; and, killing LTCs, or otherwise disrupting vital intracellular processes and/or intercellular interactions of the LTC, while effectively preserving the LTC's environment's pH & functionality.

It is another object of the invention to disclose a method as defined above, wherein the method comprising steps of providing at least one PSS; and, providing the PSS with at least one preventive barrier such that a sustained long acting is obtained.

It is another object of the invention to disclose a method as defined above, wherein the step of providing the barrier is obtained by utilizing a polymeric preventive barrier adapted to avoid heavy ion diffusion; preferably by providing the polymer as an ionomeric barrier, and particularly by utilizing a commercially available Nafion™ product.

It is hence in the scope of the invention wherein one or more of the following materials are provided: encapsulated strong acidic and strong basic buffers in solid or semi-solid envelopes, solid ion-exchangers (SIEx), ionomers, coated-SIEx, high-cross-linked small-pores SIEx, Filled-pores SIEx, matrix-embedded SIEx, ionomeric particles embedded in matrices, mixture of anionic (acidic) and cationic (basic) SIEx etc.

It is another object of the invention to disclose the PSS as defined in any of the above, wherein the PSS are naturally occurring organic acids compositions containing a variety of carbocsylic and/or sulfonic acid groups of the family, abietic acid (C₂₀H₃₀O₂) such as colophony/rosin, pine resin and alike, acidic and basic terpenes.

It is another object of the invention to disclose a method for inducing apoptosis in at least a portion of LTCs population. The method comprising steps of obtaining at least one PSS as defined in any of the above; contacting the PSS with an LTC; and, effectively disrupting the pH homeostasis and/or electrical balance within the LTC such that the LTC's apoptosis is obtained, while efficiently preserving the pH of the LTC's environment.

It is another object of the invention to disclose a method for avoiding development of LTC's resistance and selecting over resistant mutations. The method comprising steps of obtaining at least one PSS as defined above; contacting the PSS with an LTC; and, effectively disrupting the pH homeostasis and/or electrical balance within the LTC such that development of LTC's resistance and selecting over resistant mutations is avoided, while efficiently preserving the pH of the LTC's environment and patient's safety.

It is another object of the invention to disclose a method of treating a patient, comprising steps of obtaining a non-naturally occurring medical implant; providing the implant with at least one PSS as defined as defined above, adapted for disrupting pH homeostasis and/or electrical balance within an LTC; implanting the implant within a patient, or applying the same to a surface of the patient such that the implant is contacting at least one LTC; and, disrupting vital intracellular processes and/or intercellular interactions of the LTC, while effectively preserving the pH of the LTC's environment and patient's safety.

It is another object of the invention to disclose a method of treating a patient, comprising steps of administrating to a patient an effective measure of PSSs as defined above, in a manner the PSSs contacts at least one LTC; and, disrupting vital intracellular processes and/or intercellular interactions of the LTC, while effectively preserving the pH of the LTC's environment. It is in the scope of the invention wherein the PSS is administrated e.g., orally, rectally, endoscopally, brachytherapy, topically or intravenously, systemically, as a particulate matter, provided as is or by a pharmaceutically accepted carrier.

It is another object of the invention to disclose a method of regenerating a PSS as defined above; comprising at least one step selected from a group consisting of (i) regenerating the PSS; (ii) regenerating its buffering capacity; and (iii) regenerating its proton conductivity.

### BRIEF DESCRIPTION OF THE DRAWINGS

In order to understand the invention and to see how it may be implemented in practice, a plurality of preferred embodiments will now be described, by way of non-limiting example only, with reference to the accompanying drawing, in which

FIG. 1 is a graph illustrating the cytotoxic effect of the PAAG-coated silica beads against Jurkat cells as a pH and time dependent phenomena. Jurkat cells were exposed for 0, 10, 20 and 30 min to PAAG-coated silica beads. Cell viability was evaluated by LIVE/DEAD Viability Kit;

FIG. 2 is a graph illustrating the cytotoxic effect of PAAG-coated silica beads bearing different pH as a function of the beads concentration. Jurkat cells were exposed for 0, 10, 20 and 30 min to PAAG-coated silica beads. Cell viability was evaluated by LIVE/DEAD Viability Kit;

FIG. 3 is a graph illustrating the cytotoxic effect of PAAG beads against Jurkat cells as a function of beads pH and incubation time. Jurkat cells were exposed for 0, 10, 20 and 30 min to PAAG-coated silica beads. Cell viability was evaluated by LIVE/DEAD Viability Kit;

FIG. 4 is a graph illustrating the cytotoxic effect of PAAG-coated silica beads on HT-29 cells as a function of the beads pH and incubation time. HT-29 cells were exposed for 50 hrs to PAAG-coated silica beads. Cell viability was evaluated by sulforhodamine assay;

FIG. 5 is a graph illustrating the concentration-dependent cytotoxic effect of PAAG-coated silica beads on HT-29 cells. HT-29 cells were exposed for 50 hrs to different concentrations of PAAG-coated silica beads. Cell viability was evaluated by sulforhodamine assay;

FIG. 6 is a graph illustrating the cytotoxic effect of PAAG beads on HT-29 cells as a function of beads pH. HT-29 cells were exposed for 50 hrs to PAAG-coated silica beads. Cell viability was evaluated by sulforhodamine assay;

FIG. 7 is a graph illustrating the concentration-dependent cytotoxic effect of PAAG beads bearing different pH between 2 to 6, on HT-29 cells. HT-29 cells were exposed for 50 hrs to different concentrations of PAAG-coated silica beads. Cell viability was evaluated by sulforhodamine assay;

FIG. 8 is a graph illustrating the concentration-dependent cytotoxic effect of PAAG beads bearing different pH between 7 to 11, on HT-29 cells. HT-29 cells were exposed for 50 hrs to different concentrations of PAAG-coated silica beads. Cell viability was evaluated by sulforhodamine assay;

FIG. 9 is a graph illustrating a hemolytic activity of PAAG-coated silica beads. Red blood cells were exposed for 4 hrs to PAAG-coated silica beads. Hemolytic activity of the beads was detected spectrophotometrically;

FIG. 10 is a graph illustrating the cytotoxicity of PAAG-beads on Jurkat cells. Jurkat cells were exposed for 20 min to PAAG beads. Percent of live cells was evaluated by LIVE/DEAD Viability Kit;

FIG. 11 is a graph illustrating the cytotoxicity of PAAG-beads on Jurkat cells. Jurkat cells were exposed for 20 min to PAAG beads. Percent of dead cells was evaluated by LIVE/DEAD Viability Kit;

FIG. 12 is a graph illustrating PAAG-beads induce apoptosis of Jurkat cells. Jurkat cells were exposed for 20 min to PAAG beads. For detection of apoptosis, Annexin V Apoptosis Detection Kit was used;

FIG. 13 is a graph illustrating the cytotoxicity of PAAG-coated silica beads on Jurkat cells. Jurkat cells were exposed for 20 min to PAAG-coated silica beads. Percent of live cells was evaluated by LIVE/DEAD Viability Kit;

FIG. 14 is a graph illustrating the cytotoxicity of PAAG-coated silica beads on Jurkat cells. Jurkat cells were exposed for 20 min to PAAG-coated silica beads. Percent of dead cells was evaluated by LIVE/DEAD Viability Kit;

FIG. 15 is a graph illustrating PAAG-coated-silica-beads-induced apoptosis of Jurkat cells. Jurkat cells were exposed for 20 min to PAAG-coated silica beads. For detection of apoptosis, Annexin V Apoptosis Detection Kit was used;

FIG. 16 is photomicrograph illustrating morphology of control and PAAG-coated silica beads treated Jurkat cells. Cells were exposed to PAAG-coated silica beads #48 and then examined for chromatin condensation with Hoechst 33342;

FIG. 17 is photomicrograph illustrating morphology of control and PAAG-coated silica beads treated Jurkat cells. Cells were exposed to PAAG-coated silica beads #48. Morphological examination showed swollen cells with cellular blebbing, characteristic of apoptosis;

FIG. 18 is photomicrograph illustrating morphology of control and PAAG-coated silica beads treated Jurkat cells. Cells were exposed to PAAG-coated silica beads #48. Morphological examination showed swollen cells with cellular blebbing, characteristic of apoptosis;

FIG. 19 shows a concentration dependent toxicity of G1 phase cells;

FIG. 20 shows concentration dependent toxicity of G1 phase cells, and mitotic phase cells;

FIGS. 21 & 22 presents activity test on compositions A & B, respectively; and,

FIG. 23 presents tests made by PSS on Candida albicans (ATCC 10231).

### DESCRIPTION OF THE PREFERRED EMBODIMENTS

The following specification taken in conjunction with the drawings sets forth the preferred embodiments of the present invention. The embodiments of the invention disclosed herein are the best modes contemplated by the inventors for carrying out their invention in a commercial environment, although it should be understood that various modifications can be accomplished within the parameters of the present invention.

The term ‘contact’ refers hereinafter to any direct or indirect contact of a PSS with a confined volume (living target cell or virus—LTC), wherein the PSS and LTC are located adjacently, e.g., wherein the PSS approaches either the internal or external portions of the LTC; further wherein the PSS and the LTC are within a proximity which enables (i) an effective disruption of the pH homeostasis and/or electrical balance, or (ii) otherwise disrupting vital intracellular processes and/or intercellular interactions of the LTC.

The terms ‘effectively’ and ‘effectively’ refer hereinafter to an effectiveness of over 10%, additionally or alternatively, the term refers to an effectiveness of over 50%; additionally or alternatively, the term refers to an effectiveness of over 80%. It is in the scope of the invention, wherein for purposes of killing LTCs, the term refers to killing of more than 50% of the LTC population in a predetermined time, e.g., 10 min.

The term ‘additives’ refers hereinafter to one or more members of a group consisting of biocides e.g., organic biocides such as tea tree oil, rosin, abietic acid, terpens, rosemary oil etc, and inorganic biocides, such as zinc oxides, cupper and mercury, silver salts etc, markers, biomarkers, dyes, pigments, radio-labeled materials, glues, adhesives, lubricants, medicaments, sustained release drugs, nutrients, peptides, amino acids, polysaccharides, enzymes, hormones, chelators, multivalent ions, emulsifying or de-emulsifying agents, binders, fillers, thickfiers, factors, co-factors, enzymatic-inhibitors, organoleptic agents, carrying means, such as liposomes, multilayered vesicles or other vesicles, magnetic or paramagnetic materials, ferromagnetic and non-ferromagnetic materials, biocompatibility-enhancing materials and/or biodegradating materials, such as polylactic acids and polyglutaminc acids, anticorrosive pigments, anti-fouling pigments, UV absorbers, UV enhancers, blood coagulators, inhibitors of blood coagulation, e.g., heparin and the like, or any combination thereof.

The term ‘particulate matter’ refers hereinafter to one or more members of a group consisting of nano-powders, micrometer-scale powders,fine powders, free-flowing powders, dusts, aggregates, particles having an average diameter ranging from about 1 nm to about 1000 nm, or from about 1 mm to about 25 mm.

The term about' refers hereinafter to ±20% of the defined measure.

The term ‘surface’ refers hereinafter in its broadest sense. In one sense, the term refers to the outermost boundaries of an organism or inanimate object (e.g., vehicles, buildings, and food processing equipment, etc.) that are capable of being contacted by the compositions of the present invention (e.g., for animals: the skin, hair, and fur, etc., and for plants: the leaves, stems, flowering parts, seeds, roots and fruiting bodies, etc.). In another sense, the term also refers to the inner membranes and surfaces of animals and plants (e.g., for animals: the digestive tract, vascular tissues, and the like, and for plants: the vascular tissues, etc.) capable of being contacted by compositions by any of a number of transdermal delivery routes (e.g., injection, ingestion, transdermal delivery, inhalation, and the like).

It is in the scope of the invention, wherein an insoluble PSS in the form of a polymer, ceramic, gel, resin or metal oxide is disclosed. The PSS is carrying strongly acidic or strongly basic functional groups (or both) adjusted to a pH of about <4.5 or about >8.0. It is in the scope of the invention, wherein the insoluble PSS is a solid buffer.

It is also in the scope of the invention wherein material's composition is provided such that the groups are accessible to water whether they are on the surface or in the interior of the PSS. Contacting a living cell (e.g., bacteria, fungi, animal or plant cell) with the PSS kills the cell in a time period and with an effectiveness depending on the pH of the PSS, the mass of PSS contacting the cell, the specific functional group(s) carried by the PSS, and the cell type. The cell is killed by a titration process where the PSS causes a pH change within the cell. The cell is often effectively killed before membrane disruption or cell lysis occurs. The PSS kills cells without directly contacting the cells if contact is made through a coating or membrane which is permeable to water, H+ and OH− ions, but not other ions or molecules. Such a coating also serves to prevent changing the pH of the PSS or of the solution surrounding the target cell by diffusion of counterions to the PSS's functional groups. It is acknowledged in thos respect that prior art discloses cell killing by strongly cationic (basic) molecules or polymers where killing probably occurs by membrane disruption and requires contact with the strongly cationic material or insertion of at least part of the material into the outer cell membrane.

It is also in the scope of the invention wherein an insoluble polymer, ceramic, gel, resin or metal oxide carrying strongly acid (e.g. sulfonic acid or phosphoric acid) or strongly basic (e.g. quaternary or tertiary amines) functional groups (or both) of a pH of about <4.5 or about >8.0 is disclosed. The functional groups throughout the PSS are accessible to water, with a volumetric buffering capacity of about 20 to about 100 mM H⁺/l/ph pH unit, which gives a neutral pH when placed in unbuffered water (e.g., about 5<pH> about 7.5) but which kills living cells upon contact.

It is also in the scope of the invention wherein the insoluble polymer, ceramic, gel, resin or metal oxide as defined above is coated with a barrier layer permeable to water. H⁺ and OH⁻ ions, but not to larger ions or molecules, which kills living cells upon contact with the barrier layer.

It is also in the scope of the invention wherein the insoluble polymer, ceramic, gel, resin or metal oxide as defined above is provided useful for killing living cells by inducing a pH change in the cells upon contact.

It is also in the scope of the invention wherein the insoluble polymer, ceramic, gel, resin or metal oxide as defined above is provided useful for killing living cells without necessarily inserting any of its structure into or binding to the cell membrane.

It is also in the scope of the invention wherein the insoluble polymer, ceramic, gel, resin or metal oxide as defined above is provided useful for killing living cells without necessarily prior disruption of the cell membrane and lysis.

It is also in the scope of the invention wherein the insoluble polymer, ceramic, gel, resin or metal oxide as defined above is provided useful for causing a change of about <0.2 pH units of a physiological solution or body fluid surrounding a living cell while killing the living cell upon contact.

It is also in the scope of the invention wherein the insoluble polymer, ceramic, gel, resin or metal oxide as defined above is provided in the form of shapes, a coating, a film, sheets, beads, particles, microparticles or nanoparticles, fibers, threads, powders and a suspension of these particles.

Throughout the entire experimental data section, the below terminology and annotation is applicable. Unless otherwise stated, all or part of the below listed materials and compositions (see table 1 and 2) were used in the following experiments. All experiments were repeated at least two or three times.

TABLE 1
Polyacrylamide Gel (PAAG)-Coated and uncoated Silica Beads

| Serial No. | Annotation | pH |
|---|---|---|
| 1 | I | 3 |
| 2 | II | 4 |
| 3 | III | 4.5 |
| 4 | 1A | 6.5 |
| 5 | 2A | 6 |
| 6 | 3A | 6.9 |
| 7 | 4A | 5.1 |
| 8 | 5A | 5.2 |
| 9 | 6A | 5 |
| 10 | 1 | 9.5 |
| 11 | 2 | 9.8 |
| 12 | 3 | 9 |
| 13 | 4 | 10 |
| 14 | 5 | 10.5 |
| 15 | 1a | 3 |
| 16 | 1b | 3.2 |
| 17 | 1c | 3.4 |
| 18 | A | 3 |
| 19 | B | 4 |
| 20 | C | 5 |
| 21 | D | 6 |
| 22 | E | 7 |
| 23 | F | 8 |
| 24 | 1 | 9 |
| 25 | 2 | 9.5 |
| 26 | 3 | 10 |
| 27 | 4 | 10.5 |
| 28 | pH 2 | 2 |
| 29 | pH 3 | 3 |
| 30 | pH 4 | 4 |
| 31 | pH 5 | 5 |
| 32 | pH 6 | 6 |
| 33 | pH 7 | 7 |
| 34 | pH 8 | 8 |
| 35 | pH 9 | 9 |
| 36 | pH 10 | 10 |
| 37 | pH 11 | 11 |
| 48 | 2 | 2 |
| 49 | 3 | 3 |
| 50 | 4 | 4 |
| 51 | 5 | 5 |
| 52 | 6 | 6 |
| 53 | 7 | 7 |
| 54 | 8 | 8 |
| 55 | 8.3 | 8.3 |

TABLE 2
PAAG Beads

| Serial No. | Annotation | pH |
|---|---|---|
| 38 | 2 | 2 |
| 39 | 3 | 3 |
| 40 | 4 | 4 |
| 41 | 5 | 5 |
| 42 | 6 | 6 |
| 43 | 7 | 7 |
| 44 | 8 | 8 |
| 45 | 9 | 9 |
| 46 | 10 | 10 |
| 47 | 11 | 11 |

### Example 1

Cytotoxic Effect of Polyacrylamide Gel (PAAG)-Coated and Uncoated Silica Beads on Jurkat Cells

Materials and Methods

Uncoated Silica beads (˜40 nm size, Sigma, cat.#421553) in suspension and silica beads coated by photpolymerization with polyacrylamide incorporating acidic and basic acrylamido derivatives (immobilines) were stored in refrigerator +4° C. until used.

The acute T-cell leukemia Jurkat cell line, clone E6-1 (ATCC number TIB-152), was used. Jurkat cells were maintained in RPMI-1640 medium supplemented by 1 mmol sodium pyruvate, 10% FBS and penicillin-streptomycin-amphotericin (1:100).

Viability and Microscopic Observation

2 μl of beads (dilute with a 0.1% SDS solution) were added to 10⁶ Jurkat cells in 25 μl of PBS. Live/Dead Dye (LIVE-DEAD Viability Kit, Molecular Probes) was added (0.15 μl) and incubation was performed at room temperature. Cell morphology and viability was examined using a fluorescent microscope (Axioskop 2 plus; filter 4-3).

Results

Microscopic observations of Silica-beads-treated Jurkat cells were performed using Molecular Probes' LIVE/DEAD Viability Kit. This kit utilizes mixture of SYTO9 green-fluorescent nucleic acid stain and the red-fluorescent nucleic acid stain Propidium Iodide (PI). These stains differ both in their spectral characteristics and in the ability to penetrate healthy cells. SYTO9 stain generally labels cells with intact membranes and cells with damaged membranes. In contrast, PI penetrates only cells with damaged membranes, causing a reduction in the SYTO9 stain fluorescence when both dyes are present. Thus cells with damaged membranes stain fluorescent red, whereas cells with intact membranes stain fluorescent green. The fluorescence from both live and dead cells may be viewed simultaneously with standard GREEN or RED filter set.

Jurkat cells were put in contact with the functionalized Silica beads. The Beads/Jurkat-cells ratio was varied from 1:20 to 1:80, corresponding to 3×10⁶ to 0.75×10⁶ particles per one cell, respectively. Percent of dead and live cells for various groups of functionalized Silica Beads was determined by fluorescent microscopy for 7-10 random fields. Uncoated beads were used as control for these experiments.

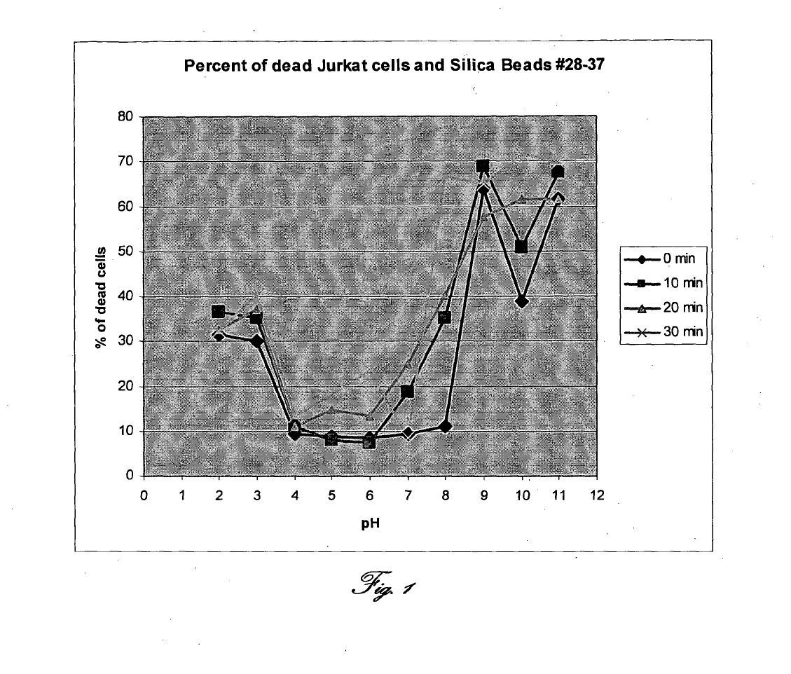

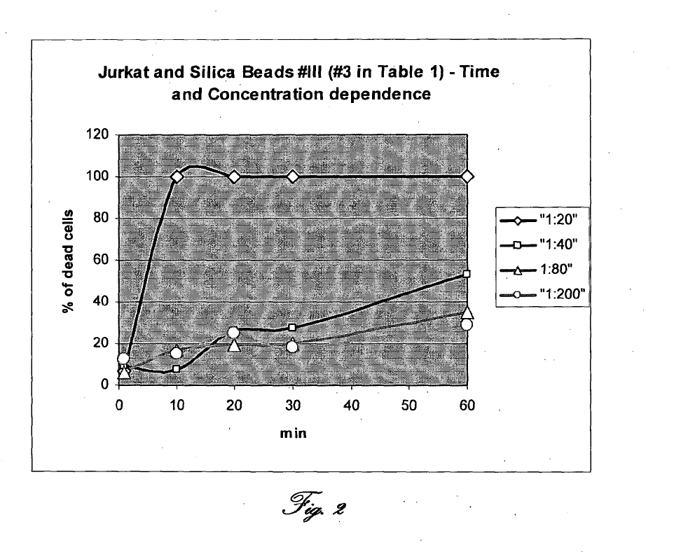

Reference is now made to FIG. 1, illustrating the pH and time dependence of the cytotoxic effect of PAAG-coated silica beads. Similarly, FIG. 2 illustrating the concentration-dependent cytotoxic effect of PAAG-coated silica beads on Jurkat cells.

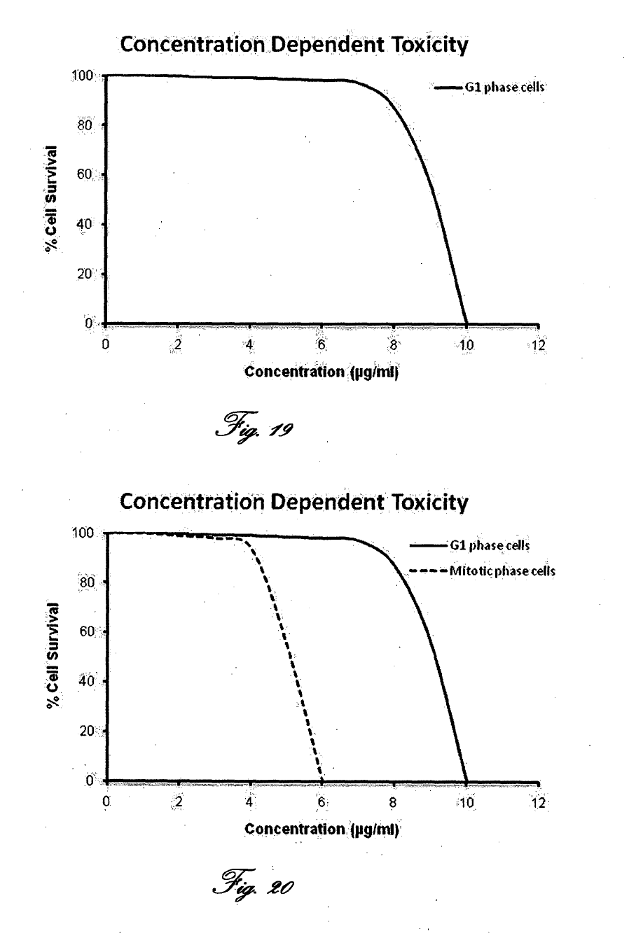

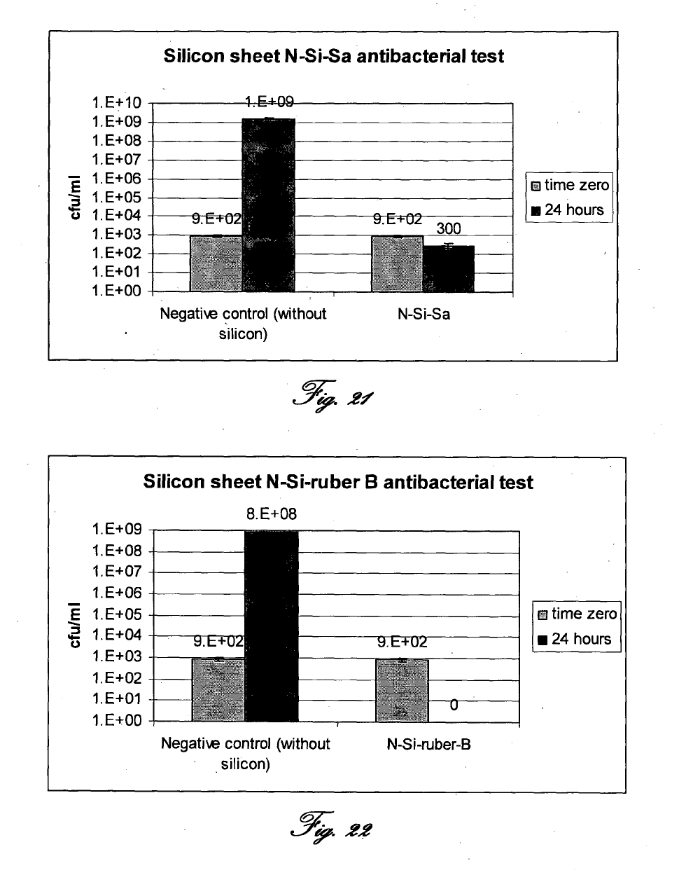

Reference is made to FIG. 19, which shows a concentration dependent toxicity of G1 phase cells; and to FIG. 20 shows concentration dependent toxicity of G1 phase cells, and mitotic phase cells. FIG. 19 presents that the % cell survival is high up to concentration of about 8 μg/ml. The PSS concentration provides an effective means of differentiation in killing LTCs. FIG. 20 illustrates two types of LTCs, wherein mitotic phase cells are killed at PSS concentration less then 5 μg/ml. In other words, at 5 μg/ml, the selectivity of the PSS towards G1 phase cells is about 2:1. Moreover, FIG. 20 demonstrates the role of PSS in differentiating between LTC and NTC, by providing a critical number of PSS' particles (or applicable surface) with a defined capacity per a given volume.

The Percentage of dead Jurkat cells in each experiment is presented in FIGS. 1 & 2. The data reveal that PAAG-coated-silica beads, carrying both strong positive and strong negative charges, exhibit high cytotoxic properties (FIGS. 1 and 2). This effect was time- and concentration-dependent (FIG. 2). Incubation of Jurkat cells with undiluted silica leads to an immediate lysis of the cells.

Acidic beads (pH2 to pH4) have lesser cytotoxic effect in comparison with basic beads. Two types of anionic substituents were assessed: substituents bearing strongly acidic sulfonic groups, which are strong, polarizable under neutral conditions and substituents bearing weakly acidic carboxyl groups for which the degree of dissociation exceeds 98% at pH˜7.

Silica beads bearing weakly acidic carboxylate substituents exhibit no cytotoxic activity compared with those of sulfonic acid substituents.

The Silica Beads bearing slight acidic, neutral and basic properties, pH from 5 to 8, seemed to be non-cytotoxic against Jurkat cells.

### Example 2

Cytotoxic Effect of PAAG Beads on Jurkat Cells

Materials and Methods

PAAG beads incorporating immobilines (size ˜500 nm) at various pH were prepared by standard emulsification techniques. Stock solutions were stored in refrigerator +4° C. until used.

The acute T-cell leukemia Jurkat cell line, clone E6-1 (ATCC number TIB-152), was used. Jurkat cells were maintained in RPMI-1640 medium supplemented by 1 mmol sodium pyruvate, 10% FBS and penicillin-streptomycin-amphotericin (1:100).

Viability and Microscopic Observation

2 μl of beads (dilute with a 0.1% SDS solution) were added to 10⁶ Jurkat cells in 25 μl of PBS. Live/Dead Dye (LIVE-DEAD Viability Kit, Molecular Probes) was added (0.15 μl) and incubation was performed at room temperature. Cell morphology and viability was examined using a fluorescent microscope (Axioskop 2 plus; filter 4-3).

Results

Microscopic observations of PAAG-beads-treated Jurkat cells were performed using Molecular Probes' LIVE/DEAD Viability Kit as described above.

Jurkat cells were put in contact with the PAAG-beads. The Beads/Jurkat-cells ratio was varied from 1:20 to 1:80, corresponding to 3×10⁶ to 0.75×10⁶ particles per one cell, respectively. Percent of dead and live cells for various groups of PAAG-Beads was determined by fluorescent microscopy for 7-10 random fields. Uncoated beads were used as control for these experiments.

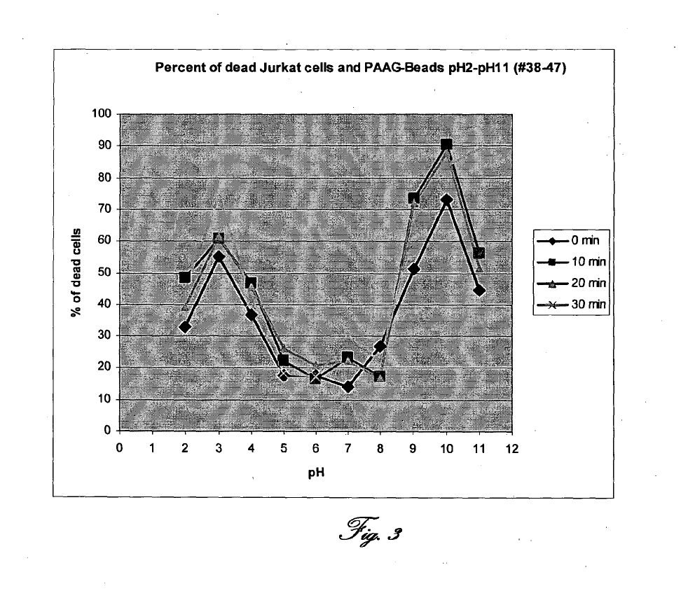

Reference is now made to FIG. 3, presenting pH and time dependence of the cytotoxic effect of PAAG beads.

The Percentage of dead Jurkat cells in this experiment is presented in FIG. 3. The data reveal that PAAG-beads, carrying both strong positive and strong negative charges, exhibit high cytotoxic properties. This effect was time- and concentration-dependent.

Acidic beads (pH2-pH4) have lesser cytotoxic effect in comparison with basic beads. Two types of anionic substituents were assessed: substituents bearing strongly acidic sulfonic groups, which are strong, polarizable under neutral conditions and substituents bearing weakly acidic carboxyl groups for which the degree of dissociation exceeds 98% at pH˜7.

### Example 3

The Cytotoxic Effect of Two Amberlite™ Beads CG-120-I and CG-400-II on Jurkat Cells

Material and Methods

Two Amberlite™ Beads CG-120-I and CG-400-II were tested for their effect on Jurkat cells: Amberlite™ CG-120-II (Fluka, 06469), strongly acidic gel-type resin with sulfonic acid functionality Na⁺ form, 200-400 mesh; and Amberlite™ CG-400-II (Fluka, 06471), strongly basic gel-type resin, quaternary ammonium functionality, Cl⁻ form, 200-400 mesh. 0.15 μl of the dye mixture (Molecular Probes' LIVE/DEAD Viability Kit) were added to 20 μl of Jurkat cells in PBS (5×105 cells). 5 μl of Amberlite™ Beads in. PBS (5×105 beads) were then added to the cells suspension. 7 μl stained cell suspension were immediately transferred to a picroscope slide and covered with a cover slip. Live and dead Jurkat cells were measured in a fluorescence microscope using 4-3 green filter.

Results

It was shown that there are no practical differences between Control and the two Amberlite™ Beads. It seems that the Na⁺ form and the Cl⁻ form possess no, cytotoxicity capabilities against Jurkat cells.

### Example 4

The Cytotoxic Effect of Two Converted Amberlite™ Beads CG-120-I and CG-400-II on Jurkat Cells

Material and Methods

The above mentioned Amberlite™ beads were converted to H+ and OH− forms according to the following procedure: Amberlite™ GC-120 (˜100 mg) were incubated in 2 ml of 0.5 M HCl at room temperature for 30 min. Amberlite™ GC-400 (˜100 mg) were incubated in 2 ml of 0.5 M NaOH at room temperature for 30 min. Beads were then washed with ˜50 ml of distilled water until the wash pH was 5 to 6 for both Amberlite™ types (GC-120 and GC-400). Stock suspension in water was prepared in a concentration of 1 mg/ml (105 beads/ml). Amberlite™ CG-120-II (Fluka, 06469), strongly acidic gel-type resin with sulfonic acid functionality H+ form, 200-400 mesh. Amberlite™ CG-400-II (Fluka, 06471), strongly basic gel-type resin, quaternary ammonim functionality, HO− form, 200-400 mesh. 0.15 μl of the dye mixture (commercially available Molecular Probes' LIVE/DEAD Viability Kit) were added to 20 μl of Jurkat cells in PBS (5×105 cells). 5 μl of Amberlite™ Beads in PBS (5×105 beads) were then added to the cells suspension. 7 μl stained cell suspension were immediately transferred to a microscope slide and covered with a cover slip. Live and dead Jurkat cells were measured in a fluorescence microscope using 4-3 green filter.

Results

The two types of converted Amberlite™ Beads CG-120-I and CG-400-II were converted to H⁺ and OH⁻ forms. Interaction of Jurkat cells with CG-400 in HO⁻ form leads to lysis of Jurkat cells; we did not observed any differences between CG-120 H⁻ form and Control.

No differences were found between CG-120 H⁺ form and Control. Interaction of Jurkat cells with CG-400 HO⁻ form leads to cell lysis.

### Example 5

The Cytotoxic Effect of PAAG-Coated Silica Beads on HT-29 Cells

Materials and Methods

PAAG-Coated and uncoated Silica beads (Sigma, cat.#421553) were prepared as described above. Stock solutions were stored in refrigerator +4° C. until used. HT-29 cells are maintained in DMEM medium supplemented by 10% FBS and penicillin-streptomycin-amphotericin (1:100).

Sulphorhodamine Cytotoxicity Test (for HT-29 Cells)

Aliquots of medium containing 1-2×10⁴ cells were distributed into a 96-well plate (Falcon). The following day, the media were replaced with 95 μl of fresh media and 5 μl of suspension containing different concentration of corresponding beads. The plate was then incubated for 72 h at 37° C. after which, 50 μl of 50% TCA were added to each well. Then after, Sulphorhodamine reagent was added and the cytotoxic effect was determined as described in the following Protocol:

First day: Add 2.5 ml/plate Trypsin-EDTA for 10 min RT (cells detachment); Transfer cells-trypsin-EDTA to 50 ml tube; Add 30 ml of DMEM/10% FCS media; Centrifuge for 10 min at 1500 rpm; Suspend cells in 20 ml of DMEM/10% FCS media; Centrifuge for 10 min 1500 rpm; Re-suspend cells in 4 ml of media; Prepare mix from X ml of cells suspension and Y ml of media; add 200 μl of cells (2×104 cells/200 μl) to each well of 96-well plate; Incubate for 24 hrs in CO2 incubator at 37° C.

Second day: Change Media and add Media and Solvent and Beads at 6 different concentrations: Add fresh medium, Solvent and Beads suspenssion; Incubate for 50 hrs in CO2 incubator at 37° C.

Third day: Wash with fresh medium five times; Add 50 μl of 50% TCA (final conc. 10% TCA); Incubate for 1 hr at 4° C.; Discard the supernatants; Wash 5 times with tap water; Invert plate and tap onto paper to remove water residuals; Let air-dry in a chemical hood over night.

Fourth day: Add 100 μl of Sulforhodamine B (0.4% w/v in 1% acetic acid); Incubate plate for 10 min at RT; Remove unbound dye by washing 5 times with 200 μl of 1% AcOH; Let the plate air-dry in a chemical hood for at least 2 hrs; Extract the dye from the cells with 200 μl of 10 mM Trizma base, pH10.3; Incubate at least 10 min at RT while shaking; Measure OD at 540 nm on a plate reader (background at 620 nm)

Results

The sulforhodamine B (SRB) assay was used for cell density determination, based on the measurement of cellular protein content. The assay relies on the ability of SRB to bind to protein components of cells that have been fixed to tissue-culture plates by trichloroacetic acid (TCA). SRB is a bright-pink aminoxanthene dye, which bind to basic amino-acid residues under mild acidic conditions, and dissociate under basic conditions. As the binding of SRB is stoichiometric, the amount of dye extracted from stained cells is directly proportional to the cell mass. The strong intensity of SRB staining allows the assay to be carried out in a 96-well format. Results from the SRB assay exhibit a linear dynamic range over densities of 7.5×10³-1.8×10⁵ cells per well, corresponding to 1-200% confluence.

The SRB assay has been developed by us for testing functionalized Beads toxicity against human HT-29 cell line (colon adenocarcinoma). To allow comparison between the different experimental conditions, the GI-50 index was expressed as the Relative Number of Beads (RNB) needed in order to induce 50% cell-growth Inhibition. In other words, the RNB value is the reciprocal to the percent of dead cells measurement used in other examples disclosed in this invention.

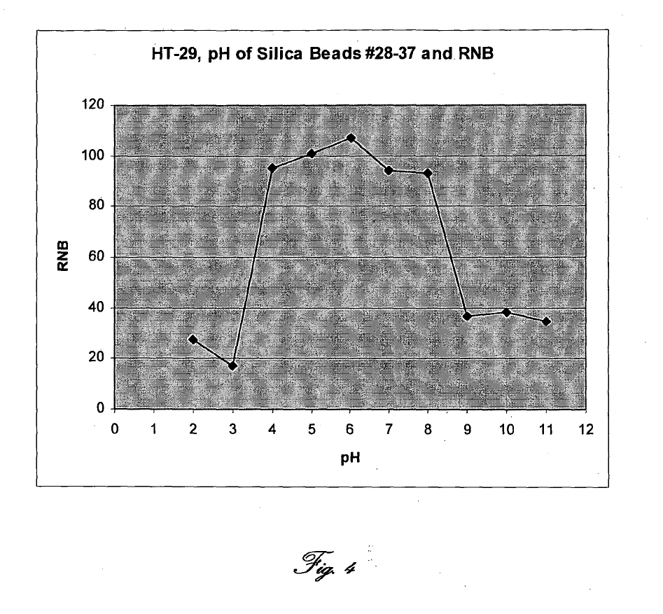

In the following experiments, HT-29 cells were put in contact with of functionalized PAAG-coated silica beads. Control experiments with uncharged beads were also systematically performed. The Beads: HT-29 cells ratio is varied from 1:20 to 1:160 or more, meaning that for each HT-29 cell there are between 156 to 19.5 million beads. SRB assay was repeated, and each concentration of Beads consisted of six to eight replicates (Table 3 and FIGS. 4 and 5).

These experiments show that PAAG-coated silica beads carrying strong acidic and strong basic groups have a cytotoxic effect on HT-29 cells. This effect is qualitatively similar to the effect observed for Jurkat cells (FIG. 1-3 above). However, a cytotoxic effect of acidic Silica Beads on the adherent HT-29 cell seems to be stronger than the effect of basic Beads.

TABLE 3
RNB as a function of PAAG-coated silica beads pH (Beads #28-37
in Table 1)

| # | pH | RNB |
|---|---|---|
| 28 | 2 | 27.2 |
| 29 | 3 | 17.2 |
| 30 | 4 | 95.2 |
| 31 | 5 | 101 |
| 32 | 6 | 107 |
| 33 | 7 | 94.3 |
| 34 | 8 | 92.9 |
| 35 | 9 | 36.6 |
| 36 | 10 | 38.5 |
| 37 | 11 | 34.7 |
| Silica | | 80 |

Reference is made to FIG. 4, illustrating the pH dependence of the cytotoxic effect of PAAG-coated silica beads on HT-29, Human adenocarcinoma cells.

Under these experimental conditions, the PAAG-coated silica beads carrying slightly acidic and basic properties seemed to be non-cytotoxic against colon HT-29 cells.

Growth inhibition of HT-29 cells by PAAG-coated silica beads is a concentration-dependent process (FIG. 5). Interaction of HT-29 cells with undiluted Silica Beads #48 (pH2) very quickly leads to lysis of the cell.

Reference is now made to FIG. 5 illustrating Concentration-dependent cytotoxic effect of PAAG-coated silica beads on HT-29 cells.

### Example 6

Cytotoxic Effect of PAAG Beads on HT-29 Cells

Materials and Methods

PAAG beads incorporating immobilines (size ˜500 nm) at various pH were prepared by standard emulsification techniques. Stock solutions were stored in refrigerator +4° C. until used. HT-29 cells are maintained in DMEM medium supplemented by 10% FBS and penicillin-streptomycin-amphotericin (1:100).

Sulphorhodamine Cytotoxicity Test (for HT-29 Cells)

Aliquots of medium containing 1-2×10⁴ cells were distributed into a 96-well plate (Falcon). The following day, the media were replaced with 95 μl of fresh media and 5 μl of suspension containing different concentration of corresponding beads. The plate was then incubated for 72 h at 37° C. after which, 50 μl of 50% TCA were added to each well. Then after, Sulphorhodamine reagent was added and the cytotoxic effect was determined according to the above described Protocol.

Results

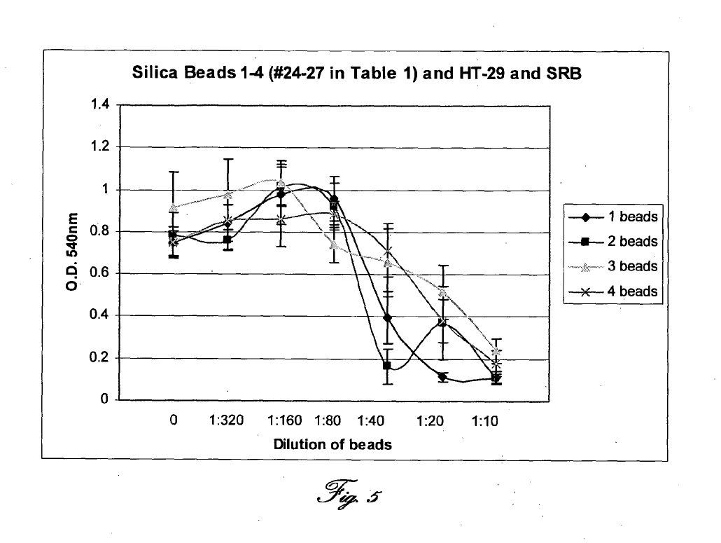

The sulforhodamine B (SRB) assay was used as described in Example 5 above. Reference is now made to FIG. 6 illustrating the pH dependence of the cytotoxic effect of PAAG-beads on HT-29, Human adenocarcinoma cells. In the following experiments, HT-29 cells were put in contact with of functionalized PAAG-beads. Control experiments with uncharged beads were also systematically performed. The Beads: HT-29 cells ratio is varied from 1:20 to 1:160 or more, meaning that for each HT-29 cell there are between 156 to 19.5 million beads. SRB assay was repeated, and each concentration of Beads consisted of six to eight replicates (FIGS. 6, 7 and 8).

These experiments show that PAAG-beads carrying strong acidic and strong basic groups have a cytotoxic effect on HT-29 cells. This effect is qualitatively similar to the effect observed for PAAG-Coated silica beads on HT-29 cells and on Jurkat cells (FIG. 1-5 above).

Under these experimental conditions, the PAAG-beads carrying slightly acidic and basic properties seemed to be non-cytotoxic against colon HT-29 cells.

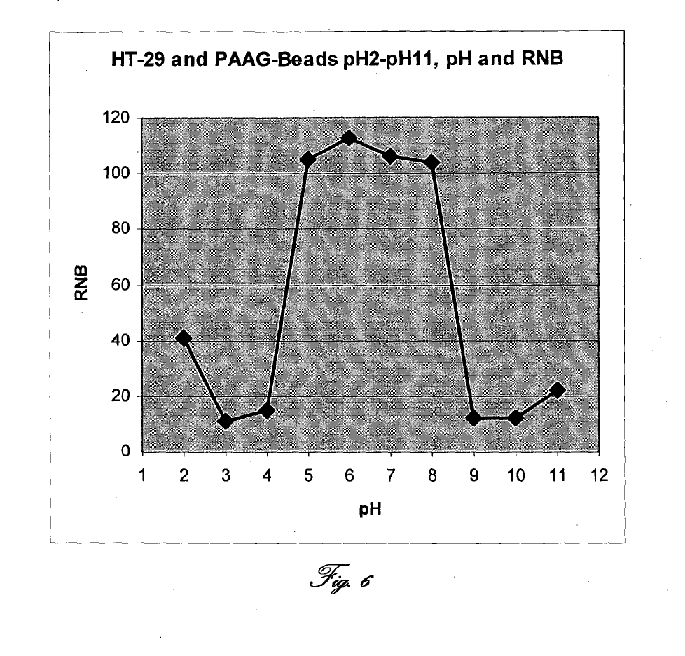

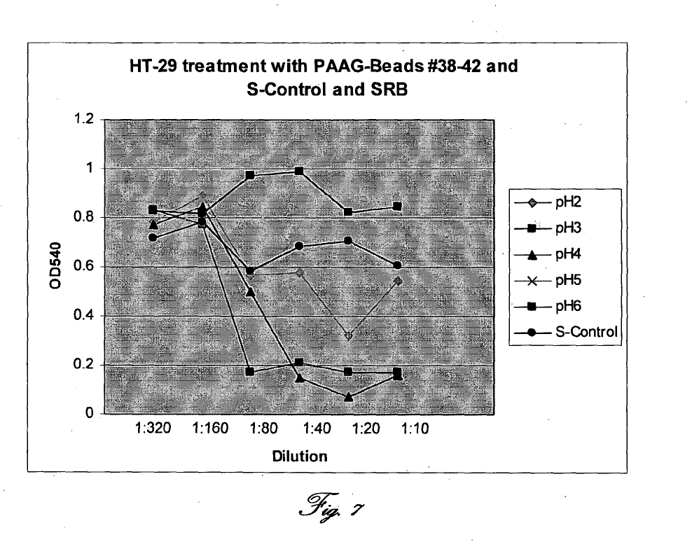

Reference is now made to FIG. 7 illustrating the pH and Concentration-dependent cytotoxic effect of PAAG-beads (pH values 2-6) on HT-29 cells; and to FIG. 8, presenting the pH and Concentration-dependent cytotoxic effect of PAAG-beads (pH values 7-11) on HT-29 cells. Growth inhibition of HT-29 cells by PAAG-beads is a concentration-dependent process (FIGS. 7 and 8). Interaction of HT-29 cells with undiluted Silica Beads #48 (pH2) very quickly leads to lysis of the cell.

### Example 7

Hemolysis Induced by PAAG-Coated of Silica Beads

Materials and Methods

Dilution of Beads: Prepare 0.2 ml of diluted beads: 10+190 μl of PBS (Ca, Mg); Preparation of RBC: Add 2 ml of blood to 13 ml of PBS; Mix gently; Centrifuge for 7 min at 2000 rpm, 10° C.; Remove the supernatant, without the RBC; Add 13 ml of PBS to the pellet and mix gently; Centrifuge as in step 3; Remove the supernatant and re-suspend the RBC in PBS to a final volume of 10 ml; Keep on ice until use.

Determination of hemolytic activity: Add 10 μl of diluted Beads to 50 μl of the washed RBC, Incubate at 37° C. with constant shaking for 4 hrs; Centrifuge the plate at 2000 rpm for 7 min at 10° C.; Transfer the supernatant to a new plate (flat bottomed) and measure absorbance at 540 nm.

Results

Reference is now to FIG. 9, presenting the role of hemolysis of RBC by PAAG-coated silica beads (see Table 1). It is shown that that all functionalized as well unmodified Silica Beads exert a strong hemolytic effect.

Dilution of Beads: Prepare 0.2 ml of diluted beads: 10+190 μl of PBS (Ca, Mg).

Preparation of RBC: Add 2 ml of blood to 13 ml of PBS; Mix gently; Centrifuge for 7 min at 2000 rpm, 10° C.; Remove the supernatant, without the RBC; Add 13 ml of PBS to the pellet and mix gently; Centrifuge as in step 3; Remove the supernatant and re-suspend the RBC in PBS to a final volume of 10 ml; Keep on ice until use; Determination of hemolytic activity; Add 10 μl of diluted Beads to 50 μl of the washed RBC; Incubate at 37° C. with constant shaking for 4 hrs; Centrifuge the plate at 2000 rpm for 7 min at 10° C.; Transfer the supernatant to a new plate (flat bottomed) and measure absorbance at 540 nm.

Results

It is shown that all functionalized as well unmodified Silica Beads exert a strong hemolytic effect (FIG. 9)

### Example 8

Apoptosis of Jurkat Cells induced by PAAG beads and PAAG-coated of Silica Beads

Materials and Methods

PAAG beads and PAAG-Coated and uncoated Silica beads (Sigma, cat. #421553) were prepared as described above. Stock solutions were stored in refrigerator +4° C. until used.

The acute T-cell leukemia Jurkat cell line, clone E6-1 (ATCC number TIB-152), was used. Jurkat cells were maintained in RPMI-1640 medium supplemented by 1 mmol sodium pyruvate, 10% FBS and penicillin-streptomycin-amphotericin (1:100).

Viability and Microscopic Observation

2 μl of beads (dilute with a 0.1% SDS solution) were added to 106 Jurkat cells in 25 μl of PBS. Live/Dead Dye (commercially available LIVE-DEAD Viability Kit, Molecular Probes) was added (0.15 μl) and incubation was performed at room temperature. Cell morphology and viability was examined using a fluorescent microscope (Axioskop 2 plus; filter 4-3).

Annexin V Apoptosis Detection Kit (Santa Cruz Biotechnology) was used for detection of apoptosis

Induction of Apoptosis-Necrosis

The following method was followed: Add 2 μl of Beads (diluted in SDS 1:30) to 20 μl (10⁶ cells) of Jurkat cells in PBS and Incubate at RT for 20 min; Collect cells by centrifugation at 2000 rpm for 3 min; Wash cell pellet with PBS and re-suspend in 1×Assay buffer at a conc. 10⁶ cells/100 μl; Add 2 μl of Annexin V FITC and 10 μl 1 of PI (Annexin V Apoptosis; Detection Kit, Santa Cruz Biotechnology); Vortex and incubate 15 min at RT in the dark; Place 10 μl of cell suspension on glass slide and cover with glass cover-slip; Use filter 4-3 or 4-4 for PI alone for microscopic examination of the results. The following controls were used: Annexin V FITC and +PI; No Annexin V FITC and no PI; Annexin V FITC alone; and PI alone.

Results

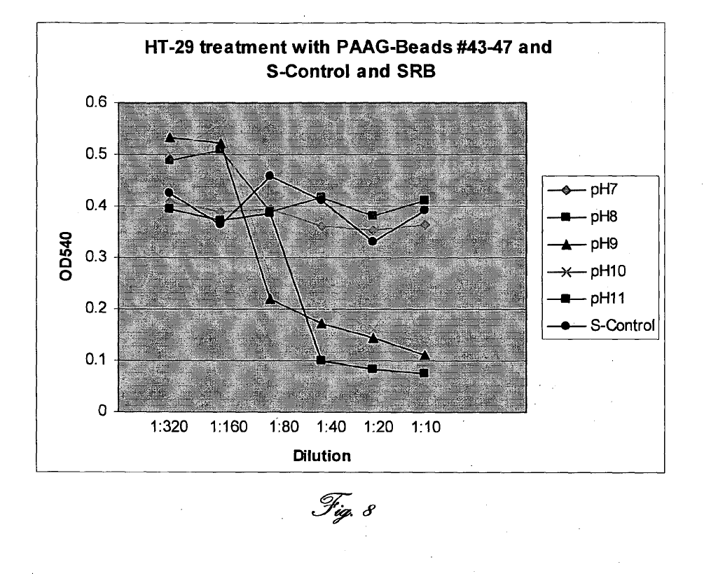

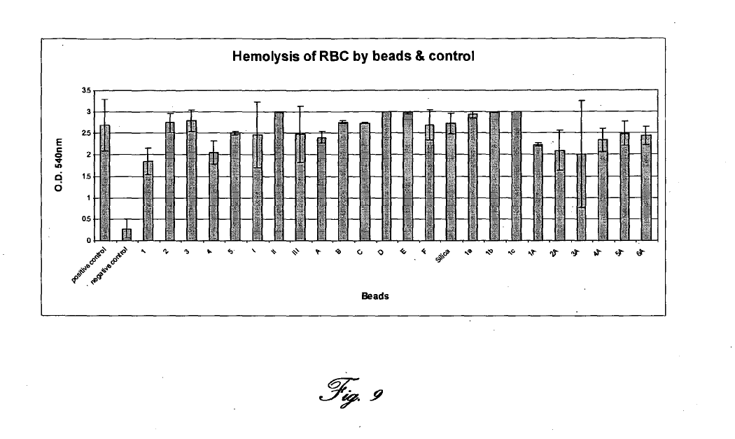

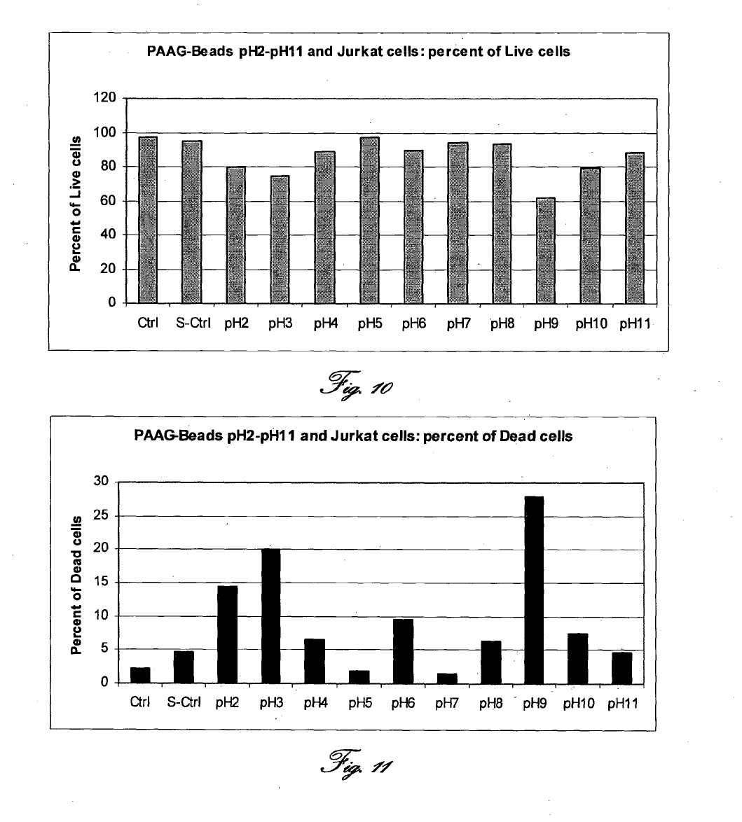

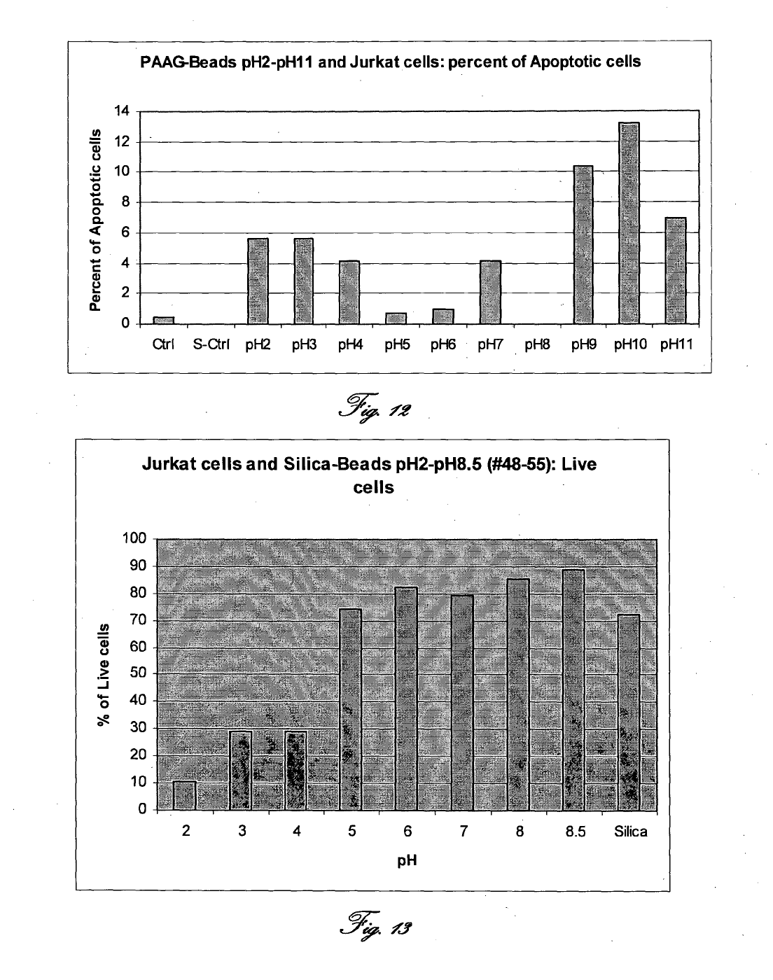

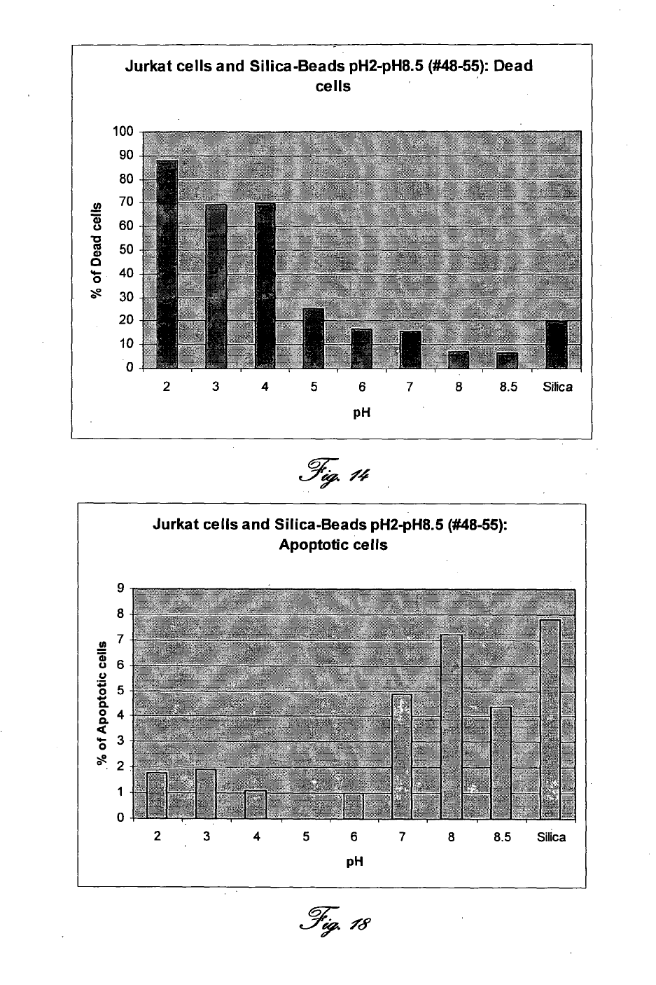

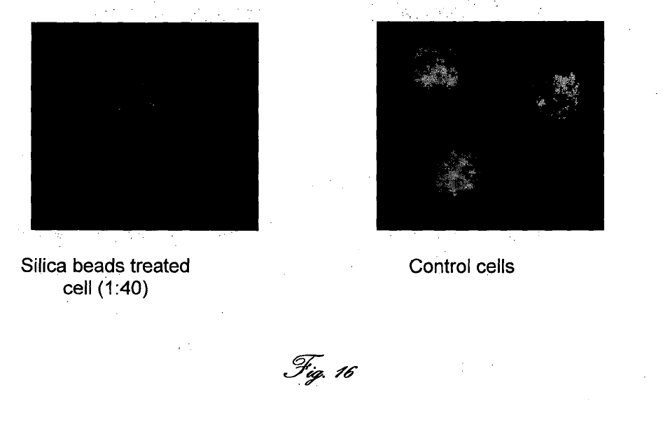

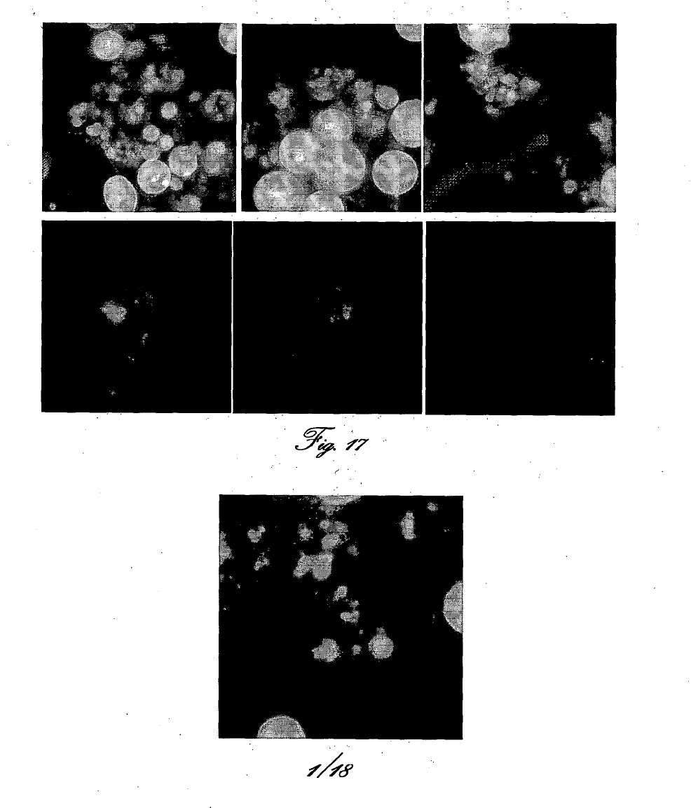

Reference is now made to FIGS. 10-18. FIG. 10 illustrates the pH induced cytotoxicity of PAAG-beads on Jurkat cells: Percentage of live cells. FIG. 11 illustrates pH induced cytotoxicity of PAAG-beads on Jurkat cells: Percentage of dead cells. FIG. 12 illustrates the pH induced apoptosis of Jurkat cells by PAAG-beads. FIG. 13 illustrates the pH induced cytotoxicity of PAAG-coated silica beads on Jurkat cells: Percentage of live cells. FIG. 14 illustrates the pH induced cytotoxicity of PAAG-coated silica beads on Jurkat cells: Percentage of dead cells. FIG. 15 illustrates the pH induced apoptosis of Jurkat cells by PAAG-coated silica beads. FIG. 16 illustrates Jurkat cells staining with Hoechst 33342 reagent after incubation with PAAG-coated silica beads pH-2 (#48 in Table 1) for 5 min. FIG. 17 illustrates Jurkat cells staining with Annexin V-PI and Dead/Live Dye after incubation with PAAG-coated silica beads pH-2 (#48 in Table 1) for 30 min. FIG. 18 is showing Jurkat cells staining with Annexin V-PI and Dead/Live Dye after incubation with PAAG-coated silica beads pH-2 (#48 in Table 1) for 90 min.

The presence of early apoptotic cells (limited nuclear fragmentation and green appearance) has been demonstrated after treatment with Silica Beads #3 (pH4.5) and #48 (pH2) and PAAG Beads #45-47 (pH 9 to pH 11). On the other hand, late apoptosis with characteristic nuclear fragmentation is also observed after treatment of Jurkat cells with Silica Beads #48 (Table 4 and FIGS. 10-18).

TABLE 4
pH induced cytotoxicity and apoptosis of Jurkat cells by PAAG-coated
silica-beads pH 2-pH 8.5 (#48-55)

| | Percentage | | |
|---|---|---|---|
| pH | Dead | Live | Apoptotic |
| 2 | 87.8 | 10.4 | 1.8 |
| 3 | 69.2 | 28.9 | 1.9 |
| 4 | 69.8 | 29.1 | 1.1 |
| 5 | 25.4 | 74.6 | 0 |
| 6 | 16.5 | 82.5 | 4.9 |
| 7 | 15.8 | 79.3 | 4.9 |
| 8 | 7.2 | 85.5 | 7.2 |
| 8.5 | 6.7 | 88.9 | 4.4 |
| Silica | 19.6 | 72.5 | 7.8 |

### Example 9

Modulation of the pH-Derived Cytotoxicity by Impregnation and Coating of Acidic and Basic Ion Exchange Beads

Experiment 1

The objective of this example was to show that by impregnation and coating of acidic and basic ion exchange beads with a neutral water permeable polymer which creates an ion selective barrier and slows down the ion exchange process the antibacterial property is enhanced.

Material and Methods

Commercial ion exchange materials: Amberlite™ CG-400-II beads (OH⁻-form) and Amberlite™ IR-120 II beads (H+-form) (Rohm and Haas, bead size ˜100 microns) were impregnated with 20% polyacrylamide.

Those beads were deposited on an agar plate inoculated with S. aureus and the antibacterial toxicity was estimated by the halo radius generated around the beads after 24 hours of incubation at 37° C.

A control experiment was performed with non treated beads.

Results

The radius of the halo around coated beads was twice as big as compared with the halo around the uncoated beads (1 min versus 0.5 mm, respectively)

Experiment 2

The objective of this example was to demonstrate that pH-derived bacterial toxicity of the materials and compositions of the current invention can be enhanced by impregnation of ion exchange beads with ionomeric polymers.

Material and Methods

Commercial ion exchange materials and Amberlite™ IR-120 II beads (H-form) (Rohm and Haas, bead size ˜100 microns) were impregnated with commercial Nafion™ (Dupont) solution and left to dry and polymerize inside the porous matrix of the ion exchange resin.

Beads obtained by this manner were deposited on an agar plate inoculated with S. aureus and the antibacterial toxicity was estimated by the halo radius generated around the beads after 24 hours of incubation at 37° C. A control experiment was performed with non treated beads. A control experiment was performed with non treated beads.

Results

The results were that the halo radius around the Nafion™-coated beads was more than 4-times as bigger as compared with that of the uncoated beads (3 mm versus 0.7 mm, respectively).

Conclusions

The experimental data disclosed in the present invention demonstrate and provide evidence for the herein proposed principal mechanism for killing cells based on preferential proton and/or hydroxyl-exchange between the cell and strong acids and/or strong basic materials and compositions. The materials and compositions of the present invention exert their cell killing effect via a titration-like process in which the the cell is coming into contact with strong acids and/or strong basic buffers and the like.

This principal mechanism was tested and found effective against both Jurkat cells which are growing in suspension and against adherent HT-29 cells as well as against bacterial cells.

The cytotoxic effects of the materials and compositions of the current invention were found to be pH, time and concentration-dependent processes; the use of the strong charged Silica Beads at final dilution 1:20 leads to an immediate lysis of the Jurkat and HT-29 cells. This effect was also evident in the Interaction of Jurkat cells with converted Amberlite™ CG-400 in their HO⁻ form.

This pH-derived cytotoxicity can be modulated by impregnation and coating of acidic and basic ion exchange materials with polymeric and/or ionomeric barrier materials

The mechanism of action underlying the cell-killing process by the materials and compositions of the current invention involves, among other things, both early and late apoptosis of the target cells, prior to their membrane disruption and cell lysis. This observation further supports the idea that, as oppose to other materials and compositions known to the art, the materials and compositions of the current invention exert their cell killing effect via a titration-like process that leads to disruption of the cell pH-homeostasis and consequently to cell death.

### Example 10

pH Preserving Antibacterial Silicone Sheet

A silicone matrix containing a mixture of acidic and basic ion exchange beads was prepared. The composition contained Amberlite™ 1200IRA (OH− form) 40% (Rohm and Haas) and Amberlite IR 120 (H+ form) 60% (Rohm and Haas). This mixture of ion exchange beads was incorporated in an inert silicon rubber solution at ratio of 40% silicon rubber (GE) and 60% Amberlite™ mixture, deposited on the inner surface of small glass jar and polymerized at 80 deg C. for 12 hours.

The antibacterial activity of the coated jars was tested as follows: An input concentration of E. coli bacteria of 660 cfu/ml was prepared. 5 ml of TSB+E. coli bacteria were added into a jar. After 24 hours the jars were sampled and decimal diluted spread on TSA plates. After 24 hours of incubation at 30° C. colonies were counted.

Results

TABLE 5
Antibacterial activity of “NEUTRAL”

| Material | cfu/ml |
|---|---|
| “NEUTRAL” | 3700 |
| Control (w/o coating) | >10¹⁰ |

pH value was equal to 7 in the tube with antibacterial material “NEUTRAL”.

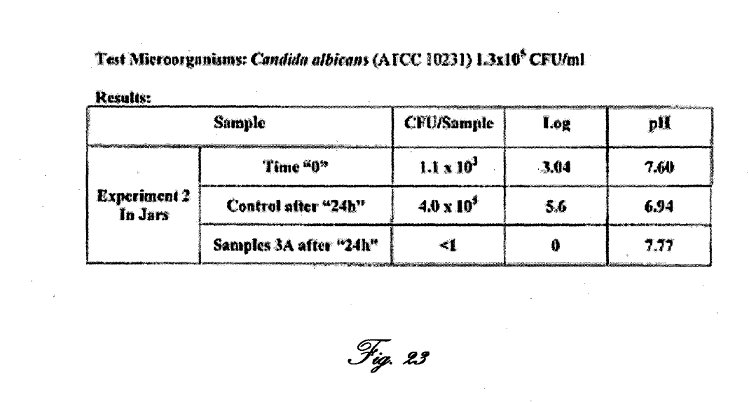

Reference in now made to FIGS. 21 and 22, presenting Activity tests on Composition A and B, respectively.

For leaching experiment, 100 mg of antibacterial material “NEUTRAL” was added to 5 ml of sterile water. Incubation was performed 48 hrs at 30° C. Potassium ions, silicone ions, sodium ions and sulfate ions were determined by ICP method.

TABLE 6
Leaching (mg/l): Exp. from 18.03.08 #1440308

| Elements | Leaching (mg/l) |
|---|---|
| S | 1.15 |
| Si | <0.002 |
| Na | 0.32 |
| K | 0.29 |

The results of table 6 show negligible release of materials from the coatings.

### Example 11

Non Leaching Bioactive Polymer (Suflon™)

A composite acidic polymer was synthesized by the following method:

Teflon (tetrafluoroethylene) monomer in n octane (20%) emulsion (CAS [116-14-3] Du Pont) was mixed with of random cross linked polystyrene sulfonate in acid form solution (27%) (Sigma Cat. No. 659592-25 ML) in n-hexane (Frutarom, Israel).

The mixture was deposited in ratio of in an autoclave and copolymerized at 50° C. and pressure of 10 atmospheres.

The resulting solution was sedimented by 0.1% of SDS (sodium dodecyl sulfate) and pressed into 0.5 mm thick sheets.

The antibacterial effect of the polymer on the growth of E. coli bacteria was tested as follows:

A 40 mg fragment of the active polymer was deposited in a 1 ml of diluted bacteria (1.E+04 cfu/ml) in TSB. The Control tube contains only bacteria in TSB. Tubes are kept in Orbital shaker at 30° C. for 24 hrs, and then are sampled for the cfu and pH measurement.

The results are as follows:

TABLE 7
Antibacterial activity of Suflon TM

| Samples | cfu/ml |
|---|---|
| SUflon | 4 × 10⁴ |
| Control | 3.1 × 10⁸ |

The results indicate inhibition of 4 logs in the presence of Suflon™ on the proliferation of E. coli bacteria.

For leaching experiment, 5 ml of sterile water (Control) and 40 mg of Suflon™ in 5 ml of sterile water are incubated at 30° C. for 24 hrs in 15-ml polypropylene tubes. These two water samples were analyzed by the ICP MS method by Spectrolab Ltd (IL).

TABLE 8
ICP analysis

| Samples | Elements | mg/l |
|---|---|---|
| Control (#1) | Na | <0.001 |
| (pH 7) | K | 0.011 |
| | S | <0.001 |
| Suflon TM (#2) | Na | <0.001 |
| (pH 7) | K | 0.018 |
| | S | <0.001 |

The results show negligible release of materials from the polymer matrix.

ICP analysis showed that Na, K and S were not found in the water containing the active polymer sample proving that the polymer composition does not leach any ingredients.

### Example 12

Antibacterial Activity of Silicone Sheets

Two types of silicone resins exhibiting bactericidal activity were prepared:

Composition A 10% 2-phenyl-5-benzidazole-sulfonic acid (Sigma 437166 25 ml); 5% Poly(styrene ran-ethylene), sulfonated, (Sigma 659401-25 ML); 80% Siloprene LSR 2060 (GE); 5% plasticizer RE-AS-2001 (MFK Inc). The mixture was spread on glass plates (thickness 1 g/10 cm**2) and polymerized at 200 deg C. for 3 hours. The polymerized sheets were peeled of the glass and tested

Composition B 15% 2-phenyl-5-benzimiddazole-sulfonic acid (Sigma 437166-25 ml) 80% Siloprene LSR 2060 (GE); 5% plastificator RE-AS-2001; The mixture was spread on glass plates (thickness 1 g/10 cm*2) and polymerized at 200° C. for 3 hours. The polymerized sheets were peeled of the glass and tested.

E. coli culture was grown overnight and was diluted 1:10⁴. 100 mg of the Silicon Sheet of Composition A and Composition B were cut and kept in Eppendorf tubes. 1 ml of the diluted culture were added the tubes. Tubes were kept rotating at room temperature and were sampled at time zero & 24 hours. Samples were decimaly diluted and were seeded on TSA plates, colonies were counted 24 hours later.

For leaching experiments 100 mg pieces of the silicone sheets of Composition A and B were placed in 5 ml of sterile water. Incubation was performed 48 hrs at 30° C. K, Na, S and Si were determined by ICP method.

TABLE 9
ICP analysis (change) Composition A

| Samples | Elements | mg/l |
|---|---|---|
| Control (#1) | Na | 0.007 |
| (pH 7) | K | 0.002 |
| | S | <0.002 |
| | Si | 0.022 |
| Silicone coating | Na | 0.027 |
| (pH 7) | K | 0.016 |
| | S | 0.006 |
| | Si | 2.238 |

TABLE 2
ICP analysis Composition B

| Samples | Elements | mg/l |
|---|---|---|
| Control (#1) | Na | 1.49 |
| (pH 7) | K | 0.056 |
| | S | 0.66 |
| | Si | 0.13 |
| Silicone coating | Na | 0.81 |
| (pH 7) | K | 0.01 |
| | S | 0.07 |
| | Si | 0.009 |

The results show negligible release of materials from the coatings.

Reference is made to FIGS. 21 & 22, presenting activity test on compositions A & B, respectively. FIG. 23 presents tests microorganisms for Candida albicans (ATCC 10231).

Hence, those PSS systems display high effectively in killing bacteria, while negligible leaching and pH change are obtained in the LTC environment.

### Example 13

Regeneration of Biocidic Activity of PSS-Containing Silicone Sheets

Two types of silicone resins exhibiting bactericidal activity were prepared. An effective measure of acid, here, ascorbic acid (Vitamin C) was utilized together with a of an ion exchanger comprising effective measure of sodium polystyrene sulphonate, as well as with other types PSSs. It was found that the acid regenerates the salt-form PSS by providing it with protons.

Moreover, articles of manufactures, such as bandages and packages for foodstuffs, beverages (e.g., juices), lotions, creams were provided with and effective measure of acid, and again, regeneration of the PSS activity was obtained.

### Example 14

Intercellular pH vs. Intracellular pH

Materials and Methods

The composition contained Amberlite™ 1200IRA (OH− form) 40% (Rohm and Haas) and Amberlite IR 120 (H+ form) 60% (Rohm and Haas). This mixture of ion exchange beads was incorporated in an inert silicon rubber solution at ratio of 40% silicon rubber (GE) and 60% Amberlite™ mixture, deposited on the inner surface of small glass jar and polymerized at 80° C. for 12 hours. E coli bacteria were used as defined above. Similarely, PAAG beads and PAAG-Coated and uncoated Silica beads were prepared as described above. Stock solutions were stored in refrigerator +4° C. until used. The acute T-cell leukemia Jurkat cell line, clone E6-1, was used as defined above. Jurkat cells were maintained in RPMI-1640 medium supplemented by 1 mmol sodium pyruvate, 10% FBS and penicillin-streptomycin-amphotericin (1:100). Commercially available pH-dependent dyes were used.

Results

A significant change in intracellular pH by incorporating pH indicator dyes internally into cells was demonstrated. The dyes color change was observed as intracellular pH changes.

1. An insoluble proton sink or source (PSS), useful for killing living target cells (LTCs), or otherwise disrupting vital intracellular processes and/or intercellular interactions of the LTC upon contact; the PSS comprising (i) proton source or sink providing a buffering capacity; and (ii) means providing proton conductivity and/or electrical potential; wherein the PSS is effectively disrupting the pH homeostasis and/or electrical balance within the confined volume of the LTC and/or disrupting vital intercellular interactions of the LTCs while efficiently preserving the pH of the LTCs' environment.

2. The PSS of claim 1, wherein said proton conductivity is provided by water permeability and/or by wetting, especially wherein said wetting is provided by hydrophilic additives.

3. The PSS of claim 2, wherein said proton conductivity or wetting is provided by inherently proton conductive materials (IPCMs) and/or inherently hydrophilic polymers (IHPs), especially by IPCMs and/or IHPs selected from a group consisting of sulfonated tetrafluoroethylene copolymers; sulfonated materials selected from a group consisting of silica, polythion-ether sulfone (SPIES), styrene-ethylene-butylene-styrene (S-SEBS), polyether-ether-ketone (PEEK), poly(arylene-ether-sulfone) (PSU), Polyvinylidene Fluoride (PVDF)-grafted styrene, polybenzimidazole (PBI) and polyphosphazene; proton-exchange membrane made by casting a polystyrene sulfonate (PSSnate) solution with suspended micron-sized particles of cross-linked PSSnate ion exchange resin; commercially available Nation™ and derivatives thereof.

4. The PSS of claim 1, comprising two or more, either two-dimensional (2D) or three-dimensional (3D) PSSs, each of which of said PSSs consisting of materials containing highly dissociating cationic and/or anionic groups (HDCAs) spatially organized in a manner which efficiently minimizes the change of the pH of the LTC's environment; each of said HDCAs is optionally spatially organized in specific either 2D, topologically folded 2D surfaces, or 3D manner efficiently which minimizes the change of the pH of the LTC's environment; further optionally, at least a portion of said spatially organized HDCAs are either 2D or 3D positioned in a manner selected from a group consisting of (i) interlacing; (ii) overlapping; (iii) conjugating; (iv) either homogeneously or heterogeneously mixing; and (iv) tiling the same.

5. The PSS of claim 1, wherein said PSS is effectively disrupting the pH homeostasis within a confined volume while efficiently preserving the entirety of said LTC's environment; and further wherein said environment's entirety is characterized by parameters selected from a group consisting of said environment functionality, chemistry; soluble's concentration, possibly other then proton or hydroxyl concentration; biological related parameters; ecological related parameters; physical parameters, especially particles size distribution, rehology and consistency; safety parameters, especially toxicity, otherwise LD50 or ICT50 affecting parameters; olphactory or organoleptic parameters (e.g., color, taste, smell, texture, conceptual appearance etc); or any combination of the same.

6. The PSS of claim 1, useful for disrupting vital intracellular processes and/or intercellular interactions of said LTC, while both (i) effectively preserving the pH of said LTC's environment and (ii) minimally affecting the entirety of the LTC's environment such that a leaching from said PSS of either ionized or neutral atoms, molecules or particles (AMP) to the LTC's environment is minimized.

7. The PSS of claim 1, useful for disrupting vital intracellular processes and/or intercellular interactions of said LTC, while less disrupting pH homeostasis and/or electrical balance within at least one second confined volume (e.g., non-target cells, NTC).

8. The PSS of claim 7, wherein said differentiation between said LTC and NTC is obtained by one or more of the following means (i) providing differential ion capacity; (ii) providing differential pH values; and, (iii) optimizing PSS to target cell size ratio; (iv) providing a differential spatial, either 2D, topologically folded 2D surfaces, or 3D configuration of said PSS; (v) providing a critical number of PSS′ particles (or applicable surface) with a defined capacity per a given volume; and (vi) providing size exclusion means.

9. The PSS of claim 1, wherein the PSS is naturally occurring organic acid containing carbocsylic and/or sulfonic acid groups, especially compositions selected from a group consisting of abietic acid (C₂₀H₃₀O₂) provided in colophony/rosin, pine resin, acidic and basic terpenes.

10. The PSS of claim 1, additionally comprising and effective measure of additives.

11. An article of manufacture, comprising at least one insoluble non-leaching PSS according to claim 1; said PSS, located on the internal and/or external surface of said article, is provided useful, upon contact, for disrupting pH homeostasis and/or electrical balance within at least a portion of an LTC while effectively preserving pH & functionality of said surface.

12. The article of manufacture of claim 11 is provided useful, upon contact for cell killing, having at least one external proton-permeable surface with a given functionality, said surface is at least partially composed of, or topically and/or underneath layered with a PSS, such disruption of vital intracellular processes and/or intercellular interactions of said LTC is provided, while said LTC's environment's pH & said functionality is effectively preserved.

13. The article of manufacture of claim 11, comprising a surface Methods, and one or more external proton-permeable layers, each of which of said layers is disposed on at least a portion of said surface; wherein said layer is at least partially composed of or layered with a PSS such that vital intracellular processes and/or intercellular interactions of said LTC are disrupted, while said LTC's environment's pH & said functionality is effectively preserved.

14. The article of manufacture of claim 13, comprising (i) at least one PSS; and (ii) one or more preventive barriers, providing said PSS with a sustained long activity; preferably wherein at least one barrier is a polymeric preventive barrier adapted to avoid heavy ion diffusion; further preferably wherein said polymer is an ionomeric barrier, and particularly a commercially available Nafion™.

15. The PSS of claim 1, adapted to avoid development of LTC's resistance and selection over resistant mutations.

16. A method for killing living target cells (LTCs), or otherwise disrupting vital intracellular processes and/or intercellular interactions of said LTC; said method comprising steps of
a. providing at least one PSS having (i) proton source or sink providing a buffering capacity; and (ii) means providing proton conductivity and/or electrical potential;
b. contacting said LTCs with said PSS; and,
c. by means of said PSS, effectively disrupting the pH homeostasis and/or electrical balance within said LTC while efficiently preserving the pH of said LTC's environment.

17. The method of claim 16, wherein said step (a) further comprising a step of providing said PSS with water permeability and/or wetting characteristics, in particular, wherein said proton conductivity and wetting is at least partially obtained by providing said PSS with hydrophilic additives.

18. The method of claim 16, further comprising a step of providing the PSS with inherently proton conductive materials (IPCMs) and/or inherently hydrophilic polymers (IHPs), especially by selecting said IPCMs and/or IHPs selected from a group consisting of sulfonated tetrafluoroethylene copolymers; sulfonated materials selected from a group consisting of silica, polythion-ether sulfone (SPTES), styrene-ethylene-butylene-styrene (S-SEBS), polyether-ether-ketone (PEEK), poly(arylene-ether-sulfone) (PSU), Polyvinylidene Fluoride (PVDF)-grafted styrene, polybenzimidazole (PBI) and polyphosphazene; proton-exchange membrane made by casting a polystyrene sulfonate (PSSnate) solution with suspended micron-sized particles of cross-linked PSSnate ion exchange resin; commercially available Nafion™ and derivatives thereof.

19. The method of claim 16, further comprising steps of
c. providing two or more, either two-dimensional (2D) or three-dimensional (3D) PSSs, each of which of said PSSs consisting of materials containing highly dissociating cationic and/or anionic groups (HDCAs); and,
d. spatially organizing said HDCAs in a manner which minimizes the change of the pH of the LTC's environment.

20. The method of claim 19, further comprising a step of spatially organizing each of said HDCAs in a specific, either 2D or 3D manner, such that the change of the pH of the LTC's environment is minimized.

21. The method of claim 20, wherein said step of organizing is provided by a manner selected from a group consisting of (i) interlacing said HDCAs; (ii) overlapping said HDCAs; (iii) conjugating said HDCAs; and (iv) either homogeneously or heterogeneously mixing said HDCAs; and (v) tiling the same.

22. The method of claim 16, further comprising a step of disrupting pH homeostasis and/or electrical potential within at least a portion of an LTC by a PSS, while both (i) effectively preserving the pH of said LTC's environment; and (ii) minimally affecting the entirety of said LTC's environment; said method is especially provided by minimizing the leaching of either ionized or electrically neutral atoms, molecules or particles (AMP) from the PSS to said environment.

23. The method of claim 16, further comprising steps of preferentially disrupting pH homeostasis and/or electrical balance within at least one first confined volume (e.g., target living cells, LTC), while less disrupting pH homeostasis within at least one second confined volume (e.g., non-target cells, NTC).

24. The differentiating method of claim 23, wherein said differentiation between said LTC and NTC is obtained by one or more of the following steps: (i) providing differential ion capacity; (ii) providing differential pH value; (iii) optimizing the PSS to LTC size ratio; and, (iv) designing a differential spatial configuration of said PSS boundaries on top of the PSS bulk; (v) providing a critical number of PSS′ particles (or applicable surface) with a defined capacity per a given volume; and (vi) providing size exclusion means.

25. A method for the production of an article of manufacture, comprising steps of providing an PSS as defined in claim 1; locating said PSS on top or underneath the surface of said article; and upon contacting said PSS with a LTC, disrupting the pH homeostasis and/or electrical balance within at least a portion of said LTC while effectively preserving pH & functionality of said surface.

26. The method of claim 25, further comprising steps of:
a. providing at least one external proton-permeable surface;
b. providing at least a portion of said surface with at least one PSS, and/or layering at least one PSS on top or underneath said surface; hence killing LTCs or otherwise disrupting vital intracellular processes and/or intercellular interactions of said LTC, while effectively preserving said LTC's environment's pH & functionality.

27. The method of claim 25, further comprising steps of:
a. providing at least one external proton-permeable surface with a given functionality;
b. disposing one or more external proton-permeable layers topically and/or underneath at least a portion of said surface; said one or more layers are at least partially composed of or layered with at least one PSS; and,
c. killing LTCs, or otherwise disrupting vital intracellular processes and/or intercellular interactions of said LTC, while effectively preserving said LTC's environment's pH & surface functionality.

28. The method of claim 16, comprising steps
a. providing at least one PSS; and,
b. providing said PSS with at least one preventive barrier such that a sustained long acting is obtained.

29. The method of claim 28, wherein said step of providing said barrier is obtained by utilizing a polymeric preventive barrier adapted to avoid heavy ion diffusion; preferably by providing said polymer as an ionomeric barrier; and particularly by utilizing a commercially available Nafion™ product.

30. A method for inducing apoptosis in at least a portion of LTCs population; said method comprising steps of:
a. obtaining at least one PSS as defined in claim 1;
b. contacting said PSS with an LTC; and,
c. effectively disrupting the pH homeostasis and/or electrical balance within said LTC such that said LTC's apoptosis is obtained, while efficiently preserving the pH of said LTC's environment.

31. A method for avoiding development of LTC's resistance and selecting over resistant mutations, said method comprising steps of:
a. obtaining at least one PSS as defined in claim 1;
b. contacting said PSS with an LTC; and,
c. effectively disrupting the pH homeostasis and/or electrical balance within said LTC, such that development of LTC's resistance and selecting over resistant mutations is avoided, while efficiently preserving the environment of said LTC's.

32. A method of treating a patient, comprising steps of:
a. obtaining a non-naturally occurring medical implant or otherwise medical device;
b. providing said implant with at least one PSS as defined in claim 1, adapted for disrupting pH homeostasis and/or electrical balance within an LTC;
c. implanting said implant within a patient, or applying the same to a surface of said patient such that said implant is contacting at least one LTC; and,
d. disrupting vital intracellular processes and/or intercellular interactions of said LTC, while effectively preserving the pH of said LTC's environment

33. A method of treating a patient, comprising steps of
a. administrating to a patient an effective measure of PSSs as defined in claim 1, in a manner said PSSs contacts at least one LTC; and,
b. disrupting vital intracellular processes and/or intercellular interactions of said LTC, while effectively preserving the pH of said LTC's environment.

34. A method of regenerating a PSS as defined in claim 1; comprising at least one step selected from a group consisting of (i) regenerating said PSS; (ii) regenerating its buffering capacity; and (iii) regenerating its proton conductivity.
